(19) United States
(12) Patent Application Publication
(10) Pub. No.: US 2009/0123529 A1
(43) Pub. Date: May 14, 2009

(54) NUCLEIC ACID IMMUNOLOGICAL COMPOSITION FOR HUMAN METAPNEUMOVIRUS

(76) Inventor: Xiaomao Li, Toronto (CA)

Correspondence Address: SMART & BIGGAR, 438 UNIVERSITY AVENUE, SUITE 1500, BOX 111, TORONTO, ON, M5G 2K8, CA

(21) Appl. No.: 12/089,030

(22) PCT Filed: Oct. 3, 2006
(86) PCT No.: PCT/CA2006/001625

§ 371 Date: Nov. 19, 2008

Related U.S. Application Data
(60) Provisional application No. 60/722,413, filed on Oct. 3, 2005.

(51) Int. Cl. A61K 39/155; A61K 31/711; A61K 9/127; C12P 21/02; A61P 31/14

(52) U.S. Cl. 424/450; 514/44; 424/211.1; 435/69.6

(57) ABSTRACT

There is provided an immunological composition that comprises a nucleic acid vector which includes a promoter region operably linked to a coding sequence encoding the human metapneumovirus F antigen or the human metapneumovirus G antigen. The immunological composition is useful for administering to an individual to elicit an immune response to human metapneumovirus in the individual and for the generation of diagnostic reagents for hMPV.

### CROSS-REFERENCE TO RELATED APPLICATION

This application claims benefit and priority from U.S. provisional patent application No. 60/722,413, filed on Oct. 3, 2005, the contents of which are incorporated herein by reference.

### FIELD OF THE INVENTION

The present invention relates generally to human metapneumovirus immunological compositions.

### BACKGROUND OF THE INVENTION

Human metapneumovirus (hMPV) is an emerging respiratory pathogen responsible for approximately 10% of respiratory diseases (Williams et. al., 2004, N. Engl. J. Med. 350(5):443-50).

Since its initial discovery in 2001 (van den Hoogen et. al., 2001), human metapneumovirus (hMPV) has become recognized as a major cause worldwide of respiratory disease (Asuncion Mejias et. al., 2004; Peret et. al., 2003; Falsey et. al., 2003; Ebihara et. al., 2003; Freymouth et. al., 2003; Vicent et. al., 2003; Jartti et. al., 2002; Maggi et. al., 2003; Viazov et. al., 2003 and Nissen et. al., 2002). Although only discovered recently, the virus has been circulated world-wide for at least 50 years. hMPV causes upper and lower respiratory tract diseases; indeed, two recent studies assigned 12% of all lower respiratory tract and 18% of all respiratory tract illness in pediatric cohorts to hMPV infection (Williams et. al., 2004; Wilkesmann et. al., 2006). Recognition of the prominence of hMPV infections has lead to intensive study of this virus and a rapid increase in knowledge of its epidemiology, pathogenesis and genomic and viral structure. The pathogenesis and disease spectrum of hMPV resembles that of human respiratory syncytial virus (RSV) and both viruses belong to the Paramyxoviridae family. The young and the elderly are particularly vulnerable, yet, no vaccine or anti-viral treatment is currently available for hMPV.

With respect to the latter, hMPV can be divided into two major genetic and antigenic subtypes, A and B. The virus is enveloped and contains a 13 kb single negative sense RNA genome encoding eight hMPV proteins (nucleoprotein (N), phosphoprotein (P), matrix protein (M), fusion protein (F), matrix protein M2 (M2), small hydrophobic protein (SH), attachment protein (G), and RNA-dependent RNA polymerase (L)) (van den Hoogen et. al., 2001 and Biacchesi et. al., 2003). The names and biological functions of these proteins have been assigned based on analogy to human respiratory syncytial virus (hRSV) and an avian cousin of hMPV, avian MPV (aMPV; van den Hoogen et. al., 2001 and Maggi et. al., 2003). Nucleotide sequence analysis of different hMPV isolates revealed two distinct genetic clusters. While some of hMPV genes are conserved, including N, M and F, the others are cluster-specific such as the G gene.

The fusion (F) protein is conserved between the hMPV A and B subtypes with 94% sequence identity. In contrast, the sequence of the attachment (G) protein contains extensive (>40 predicted) potential sites for O-linked carbohydrates and is more divergent between the two hMPV subtypes (37% identity) than the F protein. Although the F and G proteins of hMPV and hRSV are functionally similar, sequence conservation of either protein between the two viruses is rather limited (33% identity for the F protein) (van den Hoogen et. al., 2001).

As with hRSV, the F and G proteins probably are the major protective immunogens and must be considered for inclusion in any candidate vaccine (Crowe, 1995). For this reason, incorporation of the surface glycoproteins in any hMPV vaccine will likely be essential. However, there is some controversy, based on mouse studies (Plotnicky-Gilquin et. al., 2000), about whether inclusion of the G protein of hRSV in an hRSV vaccine can contribute to exacerbation of disease when the vaccinees are subsequently naturally infected with this virus.

Although only a few years have passed since the initial elucidation of hMPV, efforts to develop hMPV vaccines have already begun. The major focus appears to be directed to developing live attenuated virus vaccines possessing deletion mutations or genetic chimeras of respiratory viruses with each component being attenuated (Biacchesi et. al., 2005, Pham et. al., 2005 and Tang et. al., 2005).

Tang et. al. (Vaccine 2005, 23 (14): 1657-67) describes construction of a chimeric attenuated parainfluenza virus type 3 (PIV3) virus expressing the fusion (F) protein of hMPV. This construct was shown to be immunogenic and protective against hMPV and attenuated against PIV3 when tested in African green monkeys.

Biacchesi et. al. (J. Virol. 2005, 79(19): 12608-13) describe removal of the hMPV attachment (G), small hydrophobic (SH) or matrix M2-2 (M2-2) genes by reverse genetic engineering. The G and M2-2 deletions appeared to attenuate the virulence of the virus without compromising immune induction or protection in African green monkeys.

Both of the above approaches involve live virus, either a chimeric virus or a recombinant attenuated virus, as the immunogenic agent in a vaccine. Although such preparations have desirable characteristics as potential vaccine candidates, live virus vaccines suffer from several disadvantages, including the inherent genetic instability of the live viruses, potential difficulty in their scale-up, and problems with their storage and administration. A live vaccine must be sufficiently attenuated so as to not cause disease in the vaccinated individual, but still sufficiently immunogenic so as to elicit protection. Such vaccines inherently possess the ability to mutate during replication in the vaccinated host, and are thus potentially genetically unstable. Additionally, virus particles can be readily inactivated by environmental conditions outside of a host cell, for example by heat or by exposure to air.

In addition to live virus, isolated protein antigens are often also used as the immunogenic agent in a vaccine. A cytotoxic T-lymphocyte, epitope-based, peptide vaccine strategy has shown promise in mice (Herd et. al., 2006). However, its efficacy in genetically diverse human populations may suffer because of the intrinsic genetic restriction of these epitopes. However, proteins are relatively unstable, sensitive to storage conditions, and can denature, often resulting in a vaccine that contains an antigen with a different conformation than found in the wildtype virus.

In addition, in past RSV vaccine trials enhanced lung diseases were observed in some individuals receiving inactivated RSV antigens. Subsequent studies suggested that this was due to the induction of an imbalanced immune response to mis-folded or denatured RSV antigen and/or the presence of impurities in the vaccine preparation. Since hMPV resembles RSV, caution should be taken in the development of a safe and effective hMPV vaccine.

Accordingly, there is a need for development of an hMPV vaccine that is immunogenic, poses little or no risk of causing disease, and yet is genetically and chemically stable.

### SUMMARY OF THE INVENTION

Nucleic acid immunization is a relatively new immunization technology developed in the early 1990's. Conventional immunization involves the injection of antigens of either protein and/or carbohydrate nature, in the form of attenuated or killed microbes or purified antigens, against which immune responses develop, including protective immune responses. Nucleic acid immunization differs from these methods in that it involves direct delivery of antigen-encoding nucleic acid, often in the form of plasmid DNA, and expression of the antigens in vivo, leading to an immune response in the immunized host.

Nucleic acid vaccines and immunological compositions are typically DNA plasmid vectors that include a coding sequence of the protein antigen of interest under control of a eukaryotic promoter, which thus provides for expression of the antigen in particular mammalian cells (Garmory et al., Genetic Vaccines and Therapy 2003, 1:2-6).

Unlike live virus vaccines, which tend to target the particular cell type that the wildtype virus normally infects, nucleic acid vaccines can be delivered to a wide variety of cell types, potentially allowing for expression of the antigen in diverse cell types and/or locations in the body, particularly in cell types or locations that would not normally be infected by the virus from which the antigen is derived. However, the level of expression of individual antigens mediated by the nucleic acid vaccines depends on the particular amino acid sequence of each antigen, and nucleic acid vaccines therefore may not necessarily be suitable for all antigens.

Advantages of nucleic acid immunization include the ease of producing large amounts of the immunological composition, the relative storage stability of the immunological composition, potential immune response enhancement via the stimulation of Toll-like receptors in the hosts by CpG motifs that may be included in the vector, the potential for induction of long-lasting immune responses, as well as the fact that humoral and cellular immune responses are generated against de novo synthesized, properly folded and modified antigens. Nucleic acid immunization is also an effective method for the identification of protective antigen(s) of infectious agents. Furthermore, the de novo expression of properly folded and modified antigens allows for elicitation of a balanced immune response with reduced risk of development of disease symptoms that can arise due to denatured or impure antigens delivered in traditional purified antigen vaccines. These features distinguish it strongly and favourably from the conventional immunization and vaccination methods, where the genetic instability and the heat labile nature of live vaccines, the potential for denaturation and/or mis-folding of isolated antigen vaccines and the intrinsic inefficiency of isolated antigen vaccines and killed microbes to induce cell-mediated immunity are just a few of the disadvantages.

In one aspect, there is provided an immunological composition comprising a nucleic acid vector, the nucleic acid vector comprising a promoter region operably linked to a coding sequence encoding the human metapneumovirus F antigen or the human metapneumovirus G antigen, and a pharmaceutically acceptable carrier. The nucleic acid vector may further include one or more enhancer elements operably linked to the promoter region, or a region encoding a signal sequence included in the coding sequence.

In another aspect, there is provided a method of eliciting an immune response to human metapneumovirus in an individual, comprising administering the immunological composition as described herein to an individual in whom an immune response is desired to be elicited.

In a further aspect, there is provided a kit or commercial package including the immunological composition as described herein and instructions for administering the immunological composition to an individual.

In still another aspect, there is provided a method for producing an antibody specific against a human metapneumovirus F antigen or a human metapneumovirus G antigen comprising administering an effective amount of the immunological composition as described herein to an individual; and isolating an antibody or an immune cell from the individual, the antibodies or immune cell specific against the human metapneumovirus F antigen or human metapneumovirus G antigen. Such antibodies or immune cells are useful in preparing a polyclonal or monoclonal antibody that is specific against the human metapneumovirus F antigen or human metapneumovirus G antigen, which antibody can be used in various methods of diagnosis or for capturing or immobilizing the human metapneumovirus F antigen, human metapneumovirus G antigen or the whole hMPV.

In yet further aspects, there is provided use of an immunological composition as described herein for eliciting an immune response to human metapneumovirus in an individual and use of an immunological composition as described herein in the manufacture of a medicament for eliciting an immune response to human metapneumovirus in an individual. In still further aspects, there is provided use of the present immunological composition for producing an antibody specific against a human metapneumovirus F antigen or a human metapneumovirus G antigen, or in the manufacture of a medicament for producing an antibody specific against a human metapneumovirus F antigen or a human metapneumovirus G antigen.

Other aspects and features of the present invention will become apparent to those of ordinary skill in the art upon review of the following description of specific embodiments of the invention.

### BRIEF DESCRIPTION OF THE DRAWINGS

In the figures, which illustrate, by way of example only, embodiments of the present invention,

FIG. 1 is a graph showing anti-hMPV neutralizing antibody titres of immune sera of cotton rats inoculated with various plasmid constructs of the present invention;

FIG. 2 is a graph showing the titres of hMPV 26583 in nasal wash following viral challenge in previously inoculated cotton rats; and

FIG. 3 is a graph showing the titres of hMPV 26583 in the lungs following viral challenge in previously inoculated cotton rats.

### DETAILED DESCRIPTION

The present immunological compositions and methods use an isolated nucleic acid vector encoding the F and/or G antigens from hMPV for expressing the F and/or G antigen in an individual to elicit an immune response to hMPV in the individual, which immune response may then provide immune protection for that individual against subsequent infection with hMPV.

Thus, there is presently provided an immunological composition that includes an isolated nucleic acid vector encoding the F antigen and/or the G antigen of hMPV and which effects expression of the relevant antigen in a cell that is of the same species as an individual in which an immune response is to be elicited.

The nucleic acid vector may be any isolated nucleic acid molecule suitable for delivering a nucleic acid sequence to eukaryotic cells that is exogenous to the cells of an individual in which an immune response is to be elicited and that is capable of being expressed in such cells, and which vector excludes a viral genome, for example, wildtype or attenuated human metapneumovirus or a chimeric virus that includes a sequence encoding the human metapneumovirus F or G antigen, which chimeric virus is capable of infecting the cells of the individual in which an immune response is to be elicited. The nucleic acid vector of the present immunological composition includes single stranded or double stranded RNA, single stranded or double stranded DNA, a plasmid, an artificial chromosome, or a cosmid. Double stranded DNA is a preferred form of the nucleic acid vector given its stability both in vivo and in vitro and its ready scale-up within prokaryotic cells. In one embodiment the nucleic acid vector is a double stranded DNA plasmid. In particular embodiments the nucleic acid vector is derived from plasmid VR-1012 or plasmid VR-1020, both of which can be obtained from Vical Inc., San Diego, Calif.

The nucleic acid vector includes a promoter region for driving expression of the F or G antigen of hMPV. As will be understood, a promoter or a promoter region is a nucleotide sequence located upstream of a coding region of a gene that contains at least the minimal necessary DNA elements required to direct transcription of the coding region, and typically includes a site that directs RNA polymerase to the transcription initiation site and one or more transcription factor binding sites. A promoter, including a native promoter may include a core promoter region, for example containing a TATA box, and it may further include a regulatory region containing proximal promoter elements outside of the core promoter that act to enhance or regulate the level of transcription from the core promoter, including enhancer elements normally associated with a given promoter.

The promoter region may be any promoter region that can direct transcription of an operably linked coding sequence in a cell of the individual in which an immune response is to be elicited. For example, without limitation, the promoter may be a constitutive cellular promoter, an inducible promoter, a cellular promoter that is active only in certain cell or tissue types (a cell-specific or tissue-specific promoter), the native viral promoter for the F or G hMPV antigen, or it may be a promoter from another virus. In some embodiments, the promoter region may be the immediate early promoter from human cytomegalovirus (CMV), the promoter region from simian virus 40 (SV40), the desmin promoter/enhancer, creatine kinase promoter, the metallothionein promoter, the 1,24-vitaminD(3)(OH)(2) dehydroxylase promoter or the Rous Sarcoma Virus long terminal repeat. In particular embodiments the promoter region includes the CMV immediate early promoter or the SV40 promoter region.

The nucleic acid vector also includes a coding sequence for the antigen that is to be expressed from the immunological composition, either the F antigen of hMPV or the G antigen of hMPV, operably linked downstream of the promoter region.

A first nucleic acid sequence is operably linked with a second nucleic acid sequence when the sequences are placed in a functional relationship. For example, a coding sequence is operably linked to a promoter if the promoter activates the transcription of the coding sequence. Operably linked sequences may be contiguous, or they may be separated by an intervening nucleic acid sequence.

It will be understood that the coding sequence that is operably linked to the promoter will include or will be operably linked to any regulatory sequences necessary for transcription and translation of the coding sequence to produce the antigen of interest, either the hMPV F antigen or the hMPV G antigen. For example, if the antigen is to be expressed as a distinct polypeptide, the coding sequence should include or be operably linked to a transcription initiation sequence, a transcription termination sequence, a start codon, and a stop codon. The coding sequence also preferably includes a ribosomal binding sequence, for example a Kozak sequence, upstream to or surrounding the start codon and downstream of the transcription initiation sequence. As will be understood, if the antigen is to be expressed as a fusion protein, then such regulatory sequences and elements may be contributed by or be operably linked to the coding sequence for the fused polypeptide in such a manner that the antigen coding sequence is in frame with the coding sequence and regulatory regions of the fusion partner.

In one embodiment the coding sequence encodes the hMPV F antigen. The hMPV F antigen refers to the fusion protein (or F protein) from human metapneumovirus, and includes derivatives, variants, including allelic variants, homologs or immunogenic fragments thereof. An immunogenic fragment is a fragment of the hMPV F antigen that is sufficient to induce a humoral or cellular immune response in an individual in which an immune response is to be elicited, and may be at least 8, at least 10, at least 12, at least 14, at least 16, at least 18, at least 20, at least 25, at least 30, at least 35, at least 40, at least 45, at least 50 amino acids in length. The immunogenic fragment should elicit an immune response in the individual to whom it is administered.

A polypeptide sequence is a “homolog” of, or is “homologous” to another polypeptide sequence if the two sequences have substantial identity over a specified region and the functional activity of the sequences is conserved (as used herein, the term “homologous” does not imply evolutionary relatedness). Two polypeptide sequences are considered to have substantial identity if, when optimally aligned (with gaps permitted), they share at least approximately 50% sequence identity, or if the sequences share defined functional motifs. In alternative embodiments, optimally aligned sequences may be considered to be substantially identical (i.e. to have substantial identity) if they share at least 60%, 70%, 75%, 80%, 85%, 90%, 95%, 96%, 97%, 98%, 99% identity over a specified region. An “unrelated” or “non-homologous” sequence shares less than 40% identity, and possibly less than approximately 25% identity, with a particular polypeptide over a specified region of homology. The terms “identity” and “identical” refer to sequence similarity between two peptides or proteins. Identity can be determined by comparing each position in the aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the ClustalW program, available at http://clustalw.genome.ad.jp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis is available through the National Center for Biotechnology Information (through the internet at http://www.ncbi.nlm.nih.gov/). As used herein, “homologous amino acid sequence” includes any polypeptide having substantial identity to hMPV F antigen, as described above, including polypeptides having one or more conservative substitutions, insertions or deletions, provided the polypeptide retains the membrane fusion function and immunogenicity of the hMPV F antigen.

A variant or derivative of the hMPV F antigen refers to an hMPV F antigen or a fragment thereof that has been modified or mutated at one or more amino acids, including point, insertion or deletion mutations, but still retains the immunogenic properties of the hMPV F antigen. A variant or derivative therefore includes deletions, including truncations and fragments; insertions and additions, for example conservative substitutions, site-directed mutants and allelic variants; and modifications, including peptoids having one or more non-amino acyl groups (q.v., sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

By analogy to the F protein of RSV, the amino acid sequence of the hMPV F antigen has been divided into the following domains or regions: the signal peptide which is cleaved off after insertion of the protein to the membrane of the virus, followed by the extracellular domain including the region responsible for the fusion activity of the protein, the transmembrane domain and finally the intracellular domain. It should be noted that the boundaries of the above-mentioned domains have not been determined empirically and that any definition of the domains as given herein is an approximation. Accordingly, a skilled person will appreciate that the boundaries of the domains, including those of particular embodiments of the immunogenic fragments detailed below, are not absolute, and may be shifted N-terminally or C-terminally within the F antigen sequence relative to the boundaries as described herein.

In one embodiment, the F antigen has the following amino acid sequence, which includes the endogenous signal peptide:

[SEQ ID NO:1]

MSWKVVIIFSLLITPQHGLKESYLEESCSTITEGYLSVLRTGWYTNVFTL
EVGDVENLTCSDGPSLIKTELDLTKSALRELKTVSADQLAREEQIENPRQ
SRFVLGAIALGVATAAAVTAGVAIAKTIRLESEVTAIKNALKTTNEAVST
LGNGVRVLATAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSGKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTGFIIVIILIAV
LGSSMILVSIFIIIKKTKKPTGAPPELSGVTNNGFIPHS

In another embodiment, the F antigen used is a truncation or deletion mutant of the full-length F antigen, including a truncation or deletion mutant missing the transmembrane domain and the intracellular domain or a truncation or deletion mutant missing the signal peptide, the transmembrane domain, and the intracellular domain. In a particular embodiment, the F antigen is a truncation or deletion mutant of the full-length F antigen and has the following amino acid sequence, which includes the signal peptide and the extracellular domain but which is missing the transmembrane domain and the intracellular domain:

[SEQ ID NO:2]

MSWKVVIIFSLLITPQHGLKESYLEESCSTITEGYLSVLRTGWYTNVFTL
EVGDVENLTCSDGPSLIKTELDLTKSALRELKTVSADQLAREEQIENPRQ
SRFVLGAIALGVATAAAVTAGVAIAKTIRLESEVTAIKNALKTTNEAVST
LGNGVRVLATAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSGKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTG

In another particular embodiment, the F antigen is a truncation or deletion mutant of the full-length F antigen and has the following amino acid sequence which includes the extracellular domain but which is missing the signal peptide, the transmembrane domain and the intracellular domain:

[SEQ ID NO:3]

LKESYLEESCSTITEGYLSVLRTGWYTNVFTLEVGDVENLTCSDGPSLIK
TELDLTKSALRELKTVSADQLAREEQIENPRQSRFVLGAIALGVATAAAV
TAGVAIAKTIRLESEVTAIKNALKTTNEAVSTLGNGVRVLATAVRELKDF
VSKNLTRAINKNKCDIDDLKMAVSFSQFNRRFLNVVRQFSDNAGITPAIS
LDLMTDAELARAVSNMPTSAGQIKLMLENRAMVRRKGFGILIGVYGSSVI
YMVQLPIFGVIDTPCWIVKAAPSCSGKKGNYACLLREDQGWYCQNAGSTV
YYPNEKDCETRGDHVFCDTAAGINVAEQSKECNINISTTNYPCKVSTGRH
PISMVALSPLGALVACYKGVSCSIGSNRVGIIKQLNKGCSYITNQDADTV
TIDNTVYQLSKVEGEQHVIKGRPVSSSFDPIKFPEDQFNVALDQVFENIE
NSQALVDQSNRILSSAEKGNTG

In other embodiments, the F antigen has 80%, 85%, 90%, 95% or 99% identity to the sequence set out in any one of SEQ ID NOS: 1 to 3.

In one embodiment the coding sequence encodes the hMPV G antigen. The hMPV G antigen refers to the attachment protein (or G protein) from human metapneumovirus, and includes derivatives, variants, including allelic variants, homologs or immunogenic fragments thereof, with these terms given the analogous meaning as described above for the F antigen. Thus, an immunogenic fragment is a fragment of the hMPV G antigen that is sufficient to induce a humoral or cellular immune response in an individual in which an immune response is to be elicited, and may be at least 8, at least 10, at least 12, at least 14, at least 16, at least 18, at least 20, at least 25, at least 30, at least 35, at least 40, at least 45, at least 50 amino acids in length. The immunogenic fragment should elicit an immune response in the individual to whom it is administered.

By analogy to the G protein of RSV, the amino acid sequence of the hMPV G antigen has been divided into the following three domains: the intracellular domain, the transmembrane domain and the extracellular domain. As above for the F antigen, it should be noted that the boundaries of the above-mentioned domains of the G antigen have not been determined empirically and that any definition of the domains as given herein is an approximation. Accordingly, a skilled person will appreciate that the boundaries of the domains, including those of particular embodiments of the immunogenic fragments detailed below, are not absolute, and may be shifted N-terminally or C-terminally within the G antigen sequence relative to the boundaries as described herein.

Furthermore, the G protein of hMPV is not as conserved as the F antigen between different viral isolates, and is thus lineage-specific. Accordingly, the present immunological composition is intended to include nucleic acid vectors that encode any of the various naturally occurring G antigen variants.

In one embodiment, the G antigen has the following amino acid sequence:

[SEQ ID NO:4]

MEVKVENIRAIDMLKARVKNRVARSKCFKNASLILIGITTLSIALNIYLI
INYTIQKTSSESEHHTSSPPTESNKEASTISTDNPDINPNSQHPTQQSTE
NPTLNPAASVSPSETEPASTPDTTNRLSSVDRSTAQPSESRTKTKPTVHT
RNNPSTASSTQSPPRATTKAIRRATTFRMSSTGKRPTTTSVQSDSSTTTQ
NHEETGSANPQASVSTMQN

In another embodiment, the G antigen has the following amino acid sequence:

[SEQ ID NO:5]

MEARVENIRAIDMFKAKMKNRIRSSKCHRNATLILIGSTAPSMALNTLLI
IDHATSKNMTKVEHCVNMPPVEPSKKTPMTSAADPNTKPNPQQATQLTTE
DSTSLAATLEDHLHTGTTPTPDATVSQQTTDEHTTLLRSTNRQTTQTTAE
KKPTRATTKKETTTRTTSTAATQTLNTTNQTSNGREATTTSARSRNNATT
QSSDQTTQAADPSSQSQHTQKSTTTTHNTDTSSPSS

In another embodiment, the G antigen used is a truncation or deletion mutant of the full-length G antigen, including a truncation or deletion mutant missing the intracellular and transmembrane domains. In a particular embodiment, the G antigen is a truncation or deletion mutant of the full-length G antigen and has the following amino acid sequence, which is missing the intracellular domain and the transmembrane domain:

[SEQ ID NO:6]

NYTIQKTSSESEHHTSSPPTESNKEASTISTDNPDLNPNSQHPTQQSTEN
PTLNPAASVSPSETEPASTPDTTNRLSSVDRSTAQPSESRTKTKPTVHTR
NNPSTASSTQSPPRATTKAIRRATTFRMSSTGKRPTTTSVQSDSSTTTQN
HEETGSANPQASVSTMQN

In another particular embodiment, the G antigen is a truncation or deletion mutant of the full-length G antigen and has the following amino acid sequence, which is missing the intracellular domain and the transmembrane domain:

[SEQ ID NO:7]

DHATSKNMTKVEHCVNMPPVEPSKKTPMTSAADPNTKPNPQQATQLTTED
STSLAATLEDHLHTGTTPTPDATVSQQTTDEHTTLLRSTNRQTTQTTAEK
KPTRATTKKETTTRTTSTAATQTLNTTNQTSNGREATTTSARSRNNATTQ
SSDQTTQAADPSSQSQHTQKSTTTTHNTDTSSPSS

In other embodiments, the G antigen has 80%, 85%, 90%, 95% or 99% identity to the sequence set out in any one of SEQ ID NOS: 4 to 7.

As mentioned above, the promoter region includes a basal promoter and possibly includes enhancer elements that normally form part of the particular promoter region. In addition, the nucleic acid vector may optionally include additional enhancer elements not normally associated with the particular promoter region, operably linked to the promoter region to enhance transcription from the promoter. A promoter and an enhancer element, including a viral enhancer, are operably linked when the enhancer increases the transcription of operably linked sequences from the promoter at levels greater than from the promoter without the operably linked enhancer. As stated above, operably linked sequences may be contiguous. However, enhancers may function when separated from promoters and thus an enhancer may be operably linked to a particular promoter but may not be contiguous with that promoter. As well, multiple copies of an enhancer element may increase the transcription levels from an operably linked promoter. Thus, the placement of the optional enhancer relative to the promoter and to the coding region may vary in location, orientation and/or number.

As will be understood, an enhancer or an enhancer element is a cis-acting sequence that increases the level of transcription of a promoter, and can function in either orientation relative to the promoter and the coding sequence that is to be transcribed, and can be located upstream or downstream relative to the promoter or the coding region of a gene.

Generally, enhancers act to increase and/or activate transcription from an operably linked promoter once bound by appropriate molecules such as transcription factors. For various enhancers which may be used, transcription factor binding sites may be known or identified by one of ordinary skill using methods known in the art, for example by DNA footprinting, gel mobility shift assays, and the like. The factors may also be predicted on the basis of known consensus sequence motifs.

Reference to increasing the transcription levels or transcriptional activity is meant to refer to any detectable increase in the level of transcription of operably linked sequences compared to the level of the transcription observed with the promoter without the operably linked enhancer, as may be detected in standard transcriptional assays, including using a reporter gene constrict.

The additional enhancer element may be any enhancer element that does not normally form part of the particular promoter used, or may be additional copies of an enhancer element that already forms part of the promoter region, provided that the enhancer functions to enhance transcriptional activity of the promoter included in the nucleic acid vector of the present immunological composition in the cells of an individual in which an immune response is to be elicited.

The additional enhancer may be a viral enhancer element, for example the CMV enhancer, SV40 enhancer or it may be an enhancer element from a eukaryotic cellular gene, for example the Alpha-Fetoprotein (AFP) enhancer or the tyrosinase enhancer. In a particular embodiment, the enhancer is the human CMV immediate early enhancer.

The nucleic acid vector may optionally further include other sequences to improve the expression of the encoded antigen. For example, inclusion of an intronic sequence downstream of the promoter but upstream of transcription initiation site can result in improved expression of an operably linked coding sequence. Thus, in various embodiments the nucleic acid further includes an intronic sequence operably linked to the promoter region and the coding sequence. In one embodiment, the intron sequence includes the intron A sequence from CMV. In another embodiment, the intron sequence includes the rabbit β-globin intron II sequence.

Another sequence that may be included in the nucleic acid vector to ensure proper transcription termination is a polyadenylation signal. Thus, in various embodiments the nucleic acid vector includes a polyadenylation signal operably linked to and downstream of the coding sequence. The polyadenylation signal may be the polyadenylation signal from SV40, from the rabbit β-globin gene, from the bovine growth hormone gene or from the human growth hormone gene. In a particular embodiment, the bovine growth hormone polyadenylation signal is included.

If the antigen is desired to be expressed and secreted from the cells of the individual in which an immune response is to be elicited, a nucleotide sequence coding for a protein signal sequence, also referred to as a leader sequence, may be included in the nucleic acid to direct secretion of the protein once expressed. As will be understood, the signal sequence is a protein sequence typically included at or near the N-terminus of a secreted protein. Thus, various embodiments of the nucleic acid include a coding region for a protein signal sequence at or towards the upstream portion of the coding sequence, the signal sequence being in frame with the remainder of the coding sequence. The signal sequence may be the native signal sequence normally associated with the hMPV F antigen, or it may be another signal sequence, for example, the signal sequence from human tissue plasminogen activator.

Although the above nucleic acid vector has been described as encoding either the hMPV F antigen or the hMPV G antigen, it will be understood that the nucleic acid vector may be designed as a single nucleic acid molecule having the features described above for each of the F and G antigens to be expressed under the control of distinct promoters which may be the same or different type of promoter, for inclusion in the present immunological composition. Alternatively, the nucleic acid vector may be designed to express the F and G hMPV antigens bicistronically from a single promoter. That is, the nucleic acid vector may be designed to allow transcription of a single mRNA that contains open reading frames for the F and G antigens with corresponding translation regulatory sequences such as ribosomal binding sites for each open frame. Alternatively, it will be understood that the present immunological composition may include two different nucleic acid molecules each as described above encoding the F antigen and the G antigen, respectively.

In addition to the nucleic acid encoding the F and/or G antigen, in some embodiments the immunological composition may further comprise an adjuvant. The adjuvant may be any substance that acts to effect stimulation of an immune response, in order to increase the effectiveness of the F and/or G antigen as an immunogen. Adjuvants are well-known in the art, and may include Freund's complete adjuvant solution, Freund's incomplete adjuvant solution, a fatty acid, a monoglyceride, a protein, a carbohydrate, aluminium oxide, a toxin, killed microbes for example Mycobacterium, ethylene-vinyl acetate copolymer, L-tyrosine, manide-oleate, or immunostimulatory nucleic acid sequences for example granulocyte macrophage colony stimulating factor (GM-CSF) and CpG motifs.

If the adjuvant is a protein, an additional nucleic acid molecule encoding for the adjuvant may be included in the immunological composition, rather than the adjuvant itself. Such a nucleic acid molecule should include the coding sequence for the adjuvant protein and any necessary regulatory sequences required for expression of the adjuvant in the cells of an individual in which an immune response is to be elicited, and any desired coding region for a signal sequence for secretion of the adjuvant protein from the cells. Such a nucleic acid molecule may encode cytokines and immunostimulatory molecules such as granulocyte macrophage colony stimulating factor (GM-CSF). Such a nucleic acid molecule may be the same nucleic acid molecule as the nucleic acid vector that encodes the hMPV F and/or G antigen, or it may be a different nucleic acid molecule.

The regulatory sequences used to control and effect expression of the adjuvant may be the same or similar to those used to effect expression of the F and/or G antigen, which will help ensure that the adjuvant has the same or similar expression profile as the antigen. The expression profile includes the expression duration and levels of the expressed protein, and the particular cells in which the protein is expressed.

Alternatively, bicistronic expression of the hMPV F or G protein and the adjuvant under control of a single promoter region can be constructed on the same plasmid vector.

Alternatively, the adjuvant may be expressed as a fusion protein with the F and/or G antigen. A skilled person will understand how to design a nucleic acid vector encoding an adjuvant protein fused to the F antigen or the G antigen to result in expression of an adjuvant/F antigen or adjuvant/G antigen fusion protein, the nucleic acid vector including the various regulatory regions required for expression of the encoded sequence, as well as a coding sequence for the fused adjuvant/antigen and any required signal sequence.

Some unmethylated oligodeoxynucleotides containing CpG motifs have been shown to be immunostimulatory in mouse, human and the other animal species. Recent human trials showed that a CpG motif significantly enhanced protective antibody response to co-administered protein antigen (Cooper, C. L., et al. (2005) AIDS 19(14): 1473-9). As described in Coban C., et al. (2005) J. Leukoc. Biol. 78(3):647-55 and in Aggarwal, P., et al. (2005) Viral Immunol. 18(1):213-23, both of which are herein incorporated by reference, CpG motifs administered as adjuvant can be incorporated to the plasmid vectors, or co-administered with the plasmid vectors. Thus, in certain embodiments of the present immunological composition, the adjuvant may be an immunostimulatory nucleic acid, either included in the above-described nucleic acid vector encoding the F and/or G antigen, or included as a separate nucleic acid molecule with or in the present immunological composition.

The above described immunological composition may be formulated in a suitable vehicle for delivery to an individual in which an immune response is to be elicited and typically includes a pharmaceutically acceptable diluent or carrier that is suitable for delivery of a nucleic acid vector to eukaryotic cells, including delivery of a nucleic acid vaccine. The immunological composition may routinely contain pharmaceutically acceptable concentrations of salt, buffering agents, preservatives and various compatible carriers or diluents. For all forms of delivery, the immunological composition may be formulated in a physiological salt solution.

The proportion and identity of the pharmaceutically acceptable carrier is determined by chosen route of administration, compatibility with a nucleic acid immunological composition and standard pharmaceutical practice. Generally, the immunological composition will be formulated using components that will not significantly cause degradation of or reduce the stability or efficacy of the nucleic acid vector to effect expression of the antigen.

To assist in uptake of the nucleic acid vector or molecules by the cells of the individual in which an immune response is to be elicited, the immunological composition can be formulated with liposomes as the carrier. As will be understood, a liposome is a lipid vesicle, for example a unilamellar vesicle or a multilamellar vesicle, having a lipid exterior and a hydrophilic or aqueous interior in which the immunological composition can be encapsulated. Liposomes and methods of manufacture are generally known, for example as described in U.S. Pat. Nos. 6,936,272 and 6,228,844, which documents are herein incorporated by reference.

In addition, it will be understood that to further assist in uptake of the nucleic acid vector or molecules by the cells of the individual in which an immune response is to be elicited, the immunological composition described herein can be combined with other carriers, including substances, formulations, technologies, particles (e.g. polymer, tungsten or gold) or devices, for example the immunological composition may include gold particles to be used with gene gun for delivery of the immunological composition to the cells of the individual.

The present immunological composition may be formulated in a form that is suitable for oral or parenteral administration. Parenteral administration includes intravenous, intraperitoneal, subcutaneous, intramuscular, transepithelial, nasal, intrapulmonary, intrathecal, and topical modes of administration. Parenteral administration may be by continuous infusion over a selected period of time.

Thus, the immunological composition may be in a form suitable for oral administration, with an inert diluent or with an assimilable carrier, for example and without limitation, in the form of ingestible tablets, buccal tablets, troches, capsules, elixirs, suspensions, syrups, wafers and the like.

Alternatively, forms of the present immunological composition suitable for injection include solutions of the immunological composition, optionally encapsulated in liposomes, in association with one or more pharmaceutically acceptable vehicles or diluents, and contained in physiologically suitable buffer solutions with a suitable pH and iso-osmotic with physiological fluids. The forms of the immunological composition suitable for injectable use also include dispersions, emulsions or microemulsions and sterile powders for the extemporaneous preparation of sterile injectable solutions or dispersions. In all cases the form must be sterile. Once reconstituted from a powder, or if in liquid form for injection, the form should be fluid to the extent that easy syringability exists. Under ordinary conditions of storage and use, these preparations may contain a preservative to prevent the growth of microorganisms, but that will not cause degradation of the nucleic acid vectors or any included adjuvant.

The above described immunological composition may be prepared using standard techniques. Methods of preparation of nucleic acid molecules and vectors are generally known, including standard cloning and amplification methods. Such techniques are described for example in Sambrook et al. ((2001) Molecular Cloning: a Laboratory Manual, 3^{rd} ed., Cold Spring Harbour Laboratory Press).

The immunological composition, in a suitable formulation, can be prepared by known methods for the preparation of pharmaceutically acceptable compositions suitable for administration to individuals, such that an effective quantity of the active substance or substances is combined in a mixture with a pharmaceutically acceptable vehicle. A person skilled in the art would know how to prepare suitable formulations. Conventional procedures and ingredients for the selection and preparation of suitable formulations are described, for example, in Remington's Pharmaceutical Sciences (Remington's Pharmaceutical Sciences, Mack Publishing Company, Easton, Pa., USA 1985) and in The United States Pharmacopeia: The National Formulary (USP 24 NF19) published in 1999.

The immunological compositions described above can be used to elicit an immune response against human metapneumovirus infection in an individual. Depending on the nature of the hMPV F antigen or hMPV G antigen that is encoded by the nucleic acid vector, the immune response may be sufficient to provide full or partial protection to the individual against hMPV infection, when exposed to hMPV. Thus, the described immunological composition may be a vaccine, and may be useful for immunizing or vaccinating an individual against hMPV.

There is also presently provided a method of eliciting an immune response to human metapneumovirus in an individual in need of protection from human metapneumovirus.

In practising the method, an effective amount of the immunological composition containing the nucleic acid vector encoding the F antigen or G antigen of hMPV is administered to an individual.

An immune response includes a humoral immune response, including the production of antibodies and expansion of B cell populations, as well as a cellular immune response, including activation of T cells in response to antigens presented on the surface of antigen presenting cells. An immune response also includes a response sufficient to provide partial or complete immunity or protection against hMPV infection, as well as generation of antibodies or activation of T cells, without providing protection against hMPV infection. Thus, eliciting an immune response includes activating the humoral immune system of the individual upon exposure to antigen, activating the cellular immune system of the individual upon exposure to antigen, priming the individual's immune system to sufficient levels so as to prevent or partially prevent infection of that individual upon exposure to infectious agent, as well as vaccinating or immunizing the individual.

The individual is any individual in which it is desired to elicit an immune response to hMPV or who may need immune protection from hMPV, including an individual who has been previously exposed to hMPV, as well as an individual who has not been exposed previously to hMPV.

An effective amount of the immunological composition is administered to the individual. The term “effective amount” as used herein means an amount effective, at dosages and for periods of time necessary to achieve the desired result, including expression of the hMPV F and/or G antigen in the individual so as to allow the individual's humoral and/or cellular immune systems to recognise and effect an immune response to the antigen or antigens. For example, for an immunological composition that includes a nucleic molecule that is a DNA plasmid, a single dose for administration may include from about 0.1 μg to about 1000 μg of plasmid DNA, or from about 0.3 μg to about 350 μg of plasmid DNA.

The effective amount to be administered to an individual can vary depending on many factors such as the pharmacodynamic properties of the immunological composition, the modes of administration, the age, health and weight of the individual, and the concentration of nucleic acid vectors within the immunological composition. One of skill in the art can determine the appropriate amount of immunological composition for administration based on the above factors. The effective amount of immunological composition can be determined empirically and depends on the maximal amount of the immunological composition that can be administered safely, and the minimal amount of the immunological composition that produces the desired result.

Effective amounts of the immunological composition can be given in multiple doses, depending on the nature of the immunization regimen. For example, an initial priming dose can be given to prime the individual's immune system, and one or more subsequent doses can be given to boost the immune response generated in response to the initial priming dose. For example, the boost dose or doses can be given from 1 week to 1 year following the priming dose, and can be given periodically, for example once every 2 weeks to 6 months.

The immunological composition may be administered to the individual using standard methods of administration. In one embodiment, the immunological composition is administered orally. In another embodiment, the immunological composition is administered parenterally. In a particular embodiment, the immunological composition is administered by injection, including intramuscular injection, and including using a gene gun.

Adjuvant can be administered along with the present immunological composition, including when the immunological composition already includes adjuvant. The amount of additional adjuvant to be administered can be determined by routine experimentation by a skilled person. For example, from about 1 mg to about 10 mg of adjuvant, preferably with from about 2 mg to about 5 mg of adjuvant can be administered with the immunological composition.

The present method can include immunization with additional immunogenic agents designed to elicit an immune response against hMPV in the individual. For example, in addition to administering the present nucleic acid immunological composition, other vaccines such as attenuated virus or purified protein antigen may be administered to the same individual if desired.

In order to determine effectiveness of the immunization regimen, the individual's ability to mount an immune response to the hMPV F and/or G antigen can be determined. For example, using standard immunoassay techniques, a skilled person will be able to test for the presence of antibody and/or T-cell response in the vaccinated individual. As will be understood, such test should be conducted at a time following vaccination sufficient to allow for the generation of antibodies and/or T-cell responses in the individual, but not so long after vaccination that these immune responses in the individual will have subsided.

The present immunological composition may be packaged as a kit or commercial package containing instructions for use of the immunological composition to vaccinate an individual against human metapneumovirus.

The present immunological compositions can be used to generate antibodies specifically directed against the F or G antigen of hMPV. Thus, there is presently provided a method for generating an antibody specific against the F or the G antigen of hMPV, which involves administering the above-described immunological composition to an animal, including a human, in which the antibody is to be generated.

An antibody is specific against a particular antigen when the antibody has a higher affinity for that antigen than for other antigens, thus having the capability of selectively recognizing and binding to the particular antigen.

The antibody generated by the present method may be polyclonal or monoclonal. Monospecific antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monospecific antibodies may also be in the form of immunoglobulin fragments, e.g., F(ab)′2 or Fab fragments. The antibodies may be of any isotype, e.g., IgG or IgA, and polyclonal antibodies may be of a single isotype or a mixture of isotypes.

An effective amount of the above-described immunological composition is administered to the animal so as to produce sufficient amounts of the F or G antigen of hMPV to elicit an antibody response in the animal to the particular antigen. In most cases, an antibody will be desired to be specific to the F antigen or G antigen, but in some cases it may be desired to raise a polyclonal antibody preparation that is specific against both the F and G antigens.

The animal may be any animal capable of producing antibodies in response to exposure to an immunogen, and may be for example a human, a mouse, a rat, a rabbit or a goat.

Once the animal has had sufficient time to express the antigen and to mount an immune response against the antigen, an antibody or an immune cell is isolated or removed from the animal, depending on whether a polyclonal or monoclonal antibody preparation is desired. Methods to produce polyclonal or monoclonal antibodies are well known in the art. For a review, see “Antibodies, A Laboratory Manual, Cold Spring Harbor Laboratory, Eds. E. Harlow and D. Lane (1988), and D. E. Yelton et al., 1981. Ann. Rev. Biochem. 50:657-680; for monoclonal antibodies, see Kohler & Milstein (1975) Nature 256:495-497.

Briefly, for making monoclonal antibodies, somatic cells from a host animal immunized with antigen, with potential for producing antibody, are fused with myeloma cells, forming a hybridoma of two cells by conventional protocol. Somatic cells may be derived from the spleen, lymph node, and peripheral blood of transgenic mammals. Myeloma cells which may be used for the production of hybridomas include murine myeloma cell lines such as MPCII-45.6TGI.7, NSI-Ag4/1, SP2/0-Ag14, X63-Ag8.653, P3-NS-1-Ag-4-1, P.sub.3 X63Ag8U.sub.1, OF, and S194/5XX0.BU.1; rat cell lines including 210.RCY3.Ag1.2.3; cell lines including U-226AR and GM1500GTGA1.2; and mouse-human heteromyeloma cell lines (Hammerling, et al. (editors), Monoclonal Antibodies and T-cell Hybridomas IN: J. L. Turk (editor) Research Monographs in Immunology, Vol. 3, Elsevier/North Holland Biomedical Press, New York (1981)).

Somatic cell-myeloma cell hybrids are plated in multiple wells with a selective medium, such as HAT medium. Selective media allow for the detection of antibody producing hybridomas over other undesirable fused-cell hybrids. Selective media also prevent growth of unfused myeloma cells which would otherwise continue to divide indefinitely, since myeloma cells lack genetic information necessary to generate enzymes for cell growth. B lymphocytes derived from somatic cells contain genetic information necessary for generating enzymes for cell growth but lack the “immortal” qualities of myeloma cells, and thus, last for a short time in selective media. Therefore, only those somatic cells which have successfully fused with myeloma cells grow in the selective medium. The unfused cells were killed off by the HAT or selective medium.

A screening method is used to examine for potential anti-F or G antigen antibodies derived from hybridomas grown in the multiple wells. Multiple wells are used in order to prevent individual hybridomas from overgrowing others. Screening methods used to examine for potential anti-F or G antigen antibodies include enzyme immunoassays, radioimmunoassays, plaque assays, cytotoxicity assays, dot immunobinding assays, fluorescence activated cell sorting (FACS), and other in vitro binding assays.

Hybridomas which test positive for anti-F or G antigen antibody are maintained in culture and may be cloned in order to produce monoclonal antibodies specific for F or G antigen. Alternatively, desired hybridomas can be injected into a histocompatible animal of the type used to provide the somatic and myeloma cells for the original fusion. The injected animal develops tumors secreting the specific monoclonal antibody produced by the hybridoma.

The monoclonal antibodies secreted by the selected hybridoma cells are suitably purified from cell culture medium or ascites fluid by conventional immunoglobulin purification procedures such as, for example, protein A-Sepharose hydroxylapatite chromatography, gel electrophoresis, dialysis, or affinity chromatography.

Such antibodies are useful as diagnostic tools, for example for use in immunoassays to detect the presence of hMPV in sample, specifically the F antigen or G antigen of hMPV, such as in a biological sample, including a sample derived from a patient suspected of being infected with hMPV, for example a blood, serum, nasal or sputum sample. The antibodies may also be useful as capture molecules for capturing hMPV or the F or G antigen of hMPV, for example as a stationary phase in affinity chromatography for isolation, purification or immobilization of the captured virus particle or antigen.

Also presently contemplated is the use of the present immunological composition for eliciting an immune response against human metapneumovirus in an individual, or the use of the present immunological composition in the manufacture of a medicament for eliciting an immune response against human metapneumovirus in an individual. As well, use of the present immunological composition for producing an antibody specific against a human metapneumovirus F antigen or a human metapneumovirus G antigen, or in the manufacture of a medicament for producing an antibody specific against a human metapneumovirus F antigen or a human metapneumovirus G antigen, is contemplated.

### EXAMPLES

### Example 1

The described study involves hMPV culture, isolation and amplification of the fusion (F) and attachment (G) genes of hMPV, and optimization of sequences encoding these antigens in the latest DNA immunization vectors. The described vectors are to be evaluated in the cotton rat models of hMPV infection.

A panel of seven DNA vectors encoding the F and G proteins of hMPV have been constructed as follows.

Two clinically representative hMPV subgroups (CDC26583=CAN97-83; CDC26575=CAN98-75) and a permissive monkey tertiary cell line, LLC-MK2, were obtained. LLC-MK2 cells were successfully infected with the two hMPV subgroups. Total RNA was isolated from the hMPV-infected LLC-MK2 cells using RNeasy kits (Qiagen).

Seven DNA immunological composition vectors were constructed using reverse transcription-polymerase chain reaction (RT-PCR) on total RNA isolated from hMPV-infected LLC-MK2 cells. These vectors were made in VR-1012 and VR-1020 obtained from Vical Inc.

VR-1012 developed by Vical Inc. has been widely used for DNA immunization, including clinical trials. It contains an expression cassette with several transcription control elements, including the immediate early (IE) promoter and intron A sequences of the human cytomegalovirus (CMV), and the poly-A signal from bovine growth hormone (bGH) gene. Gene of interest with its own initiation codon and Kozak sequence is to be cloned downstream of the CMV IE promoter and intron A, and upstream of the bGH poly-A signal. To determine feasibility of DNA immunization for hMPV, we have made four DNA vectors in VR1012, encoding the conserved F and subgroup-specific G proteins of hMPV. The following vectors were constructed using VR-1012:

VR-1012 Intact F gene (CDC26583=CAN97-83). This construct is designated Clone 5-2. It expresses a full-length, membrane anchored F protein.

VR-1012 Intact G gene (CDC26583=CAN97-83). This construct is designated Clone 2-4. It expresses a full-length, membrane anchored G protein.

VR-1012 Intact G gene (CDC26575=CAN98-75). This construct is designated Clone 3-4. It expresses a full-length, membrane anchored G protein.

VR-1012 Intact F gene (CDC26583=CAN97-83) minus the coding sequences for the trans-membrane (TM) and intracellular domains. This construct is designated Clone 11-1. It expresses a truncated, secreted version of the F protein directed by the authentic signal peptide.

VR-1020 also developed by Vical Inc. directs the expression of secreted proteins. It has transcription control elements identical to those found in VR1012. In addition, it contains coding sequences for the signal peptide of human tissue plasminogen activator (TPA) downstream of the CMV IE promoter and intron A sequences and upstream of the bGH poly-A site. Gene of interest devoid of the authentic signal peptides is to be cloned downstream of the coding sequences for the signal peptide of TPA and upstream of the bGH poly-A signal in VR-1020. This insertion has to be in frame with the TPA signal peptide, so that the latter will direct secretion of the expressed foreign protein. We have made three vectors in VR1020, encoding the conserved F and subgroup-specific G proteins of hMPV. The following vectors were constructed using VR-1020:

VR-1020 Intact F gene (CDC26583=CAN97-83) minus the coding sequences for the signal peptide, the TM domain and the intracellular domain. It expresses a truncated F protein, whose secretion is directed by the TPA signal peptide. This vector is designated Clone 7-1.

VR-1020 Intact G gene (CDC26583=CAN97-83) minus the coding sequences for the intracellular domain and the TM domain. It expresses a truncated G protein, whose secretion is directed by the TPA signal peptide. This construct is designated Clone 8-2.

VR-1020 Intact G gene (CDC26575=CAN98-75) minus the coding sequences for the intracellular domain and the TM domain. It expresses a truncated G protein, whose secretion is directed by the TPA signal peptide. This vector is designated Clone 9-1.

Nucleotide sequences of the hMPV F and G gene inserts in the VR-1012 and VR-1020 vectors were confirmed completely.

We have applied the following strategies to overcome key obstacles encountered: (i) use of the most sensitive Reverse Transcriptase on the market (i.e. Qiagen's SensiScript) to selectively amplify hMPV-specific low abundance mRNA; (ii) use of a version of Taq Polymerase that has proof-reading capability (i.e. Qiagen's ProofStart) to avoid sequence errors that could be generated if using the original Taq Polymerase.

The amino acid sequence of the F protein is conserved between the two lineages and should be cross-protective against both, whereas that for the G protein is lineage-specific and should only confer protection against the particular hMPV lineage where the gene was derived.

In the first experiment, groups of cotton rats are immunized via the intramuscular route with 100 μg of each plasmid DNA construct encoding the hMPV F or G protein three times at 3 week intervals (i.e. each animal will receive 3×100 μg of the plasmid). Three weeks post the last immunization, these cotton rats are challenged intranasally with live hMPV of both genetic lineages, respectively, and sacrificed 4 days following the viral challenge. hMPV titres are assessed in the lung (i.e. the lower respiratory tract) and nose (i.e. the upper respiratory tract) to determine susceptibility for hMPV infection. Throughout the duration of the experiment, sera are taken periodically from the animals to determine hMPV-neutralizing titres. Live hMPV infection and immunization with the empty plasmid vector (i.e. VR-1012) serve as the positive and negative controls of the study, respectively.

Subsequent animal studies for more detailed characterization of the immune and other responses elicited by plasmid DNA vectors encoding the hMPV F and G proteins as described herein may be performed. For example, lung histopathology determination may be performed in animals immunized with the plasmid vectors and challenged with live hMPV, using a group immunized and challenged with live hMPV as a control, to determine the effect of the plasmid vectors encoding the hMPV F and G proteins to cause/enhance lung disease. Such experiments are typically performed in a time frame at which maximum lung pathology would be observed in order to increase the sensitivity of the experiments, for example 7-10 days post-viral challenge.

### Example 2

This study provides further detailed description of the preparation of the vectors outlined above in Example 1 and provides details of additional experiments in which the different plasmid-based vectors capable of producing different forms of the F and G proteins of hMPV were evaluated for immunogenicity and their ability to protect the inoculated animals from upper and lower pulmonary tract hMPV infection.

Materials and Methods

Animals: Male and female cotton rats (Sigmoden hispidis) weighing between 50 and 100 g were used in these experiments. All were descendants of six pair of animals obtained in 1984 from the Small Animal Section of the Veterinary Research Branch, Division of Research Services, National Institutes of Health (NIH). These cotton rats were housed in the Baylor College of Medicine (BCM) vivarium in cages covered with barrier filters and each was given food and water ad libitum. Blood samples obtained from representative animals housed in these spaces at intervals before or during the course of these experiments were seronegative for adventitious viruses and other rodent pathogens. All of the experiments were carried out utilizing NIH and United States Department of Agriculture guidelines and experimental protocols approved by the BCM Investigational Animal Care and Use Committee (IACUC).

Tissue culture: The LLC-MK2 rhesus monkey kidney tissue culture cells utilized in these studies were purchased from the American Type Culture Collection (ATCC), Manassas, Va. (cat. no. CCL7). Eagle's minimal essential medium (MEM; Sigma Chemical Co; cat. no. M4465) supplemented with 10% fetal calf serum (FCS; Summit Biotechnology; cat. no. FP-200-05), 100 U/ml penicillin (Sigma cat. no. P-4458), 100 μg/ml gentamicin sulfate (Sigma cat. no. G-1264), 2 mM L-glutamine (Sigma cat. no. G7513) and 0.2% sodium bicarbonate (Sigma cat. no. S8761) was used to grow these cells. Similarly supplemented MEM containing 0.5 μg trypsin/ml (WT; Worthington Biochemical Corp., cat. no. 32C5468) but lacking FCS was utilized to maintain the LLC-MK2 cells when preparing pools of hMPV or when performing any assay in which hMPV was involved. The trypsin-containing medium free of FCS (MEM-FCS+WT) was utilized in conjunction with hMPV because this protease was required for optimal replication of this virus in LLC-MK2 cells.

Viruses: Seed vials of two of the hMPV strains utilized in these studies (i.e., CDC 26575 and CDC 26583) were obtained from the Centers for Disease Control (CDC), Atlanta, Ga., with permission from Dr. Guy Boivin at the Research Center in Infectious Diseases, Regional Virology Laboratory, Laval University, Quebec City, Canada. These viruses also carry the designation CAN 97-83 (=CDC 26583) and CAN 98-75 (=CDC 26575) and represent subtype A and B hMPV, respectively. Characterization and preparation of working stocks of each of these viruses in LLC-MK2 tissue culture using MEM-FCS+WT has been described in detail previously (Wyde et. al., 2003 and Wyde et. al., 2005).

Isolation of the hMPV F and G genes using RT-PCR: Each of the hMPV subtypes were used to infect monolayers of LLC-MK2 cells in 75 cm² flasks. Ten days post infection when virus-induced cytopathic effects (CPE) were extensive, total RNA was isolated from the infected cells using RNeasy Mini Kits (Qiagen, Mississauga, Ontario, CA) according to manufacturer's instruction. The isolated RNA was assessed and quantified using UV absorbance at 260 nm and 280 nm, respectively. Satisfactory A₂₆₀/A₂₈₀ ratios of 1.97-2.00 were obtained from these samples that were then divided into 5 μL aliquots and stored at −20° C. For each experiment, a fresh frozen aliquot was thawed and used to ensure integrity of the hMPV genes.

A one-step reverse transcription-polymerase chain reaction (RT-PCR) protocol was used to amplify the full length F and G genes of hMPV from the isolated RNA samples. As the F gene is well conserved between the hMPV A and B subtypes, it was decided to isolate the F cDNA from CAN 97-83 only. In contrast, cDNAs encoding the G protein from both CAN 97-83 and CAN 98-75 were isolated. Based on the published nucleotide sequences of the F and G genes in CAN97-83 and CAN98-75 (Accession No. AY297749 and AY297748), and with the intention of also introducing unique restriction sites at the ends of the RT-PCR products for convenient subsequent sub-cloning purpose, the following oligo-nucleotides were designed as the primers for the RT-PCR reactions:

Forward Primer for CAN 97-83 F Gene

[SEQ ID NO.:8]

5′ GGCGGCCGCCGTCGACAAAATGTCTTGGAAAGTGGTGATCA 3′
SalI Met
Reverse Primer for CAN 97-83 F Gene

[SEQ ID NO.:9]

5′ GGCGGG TCTAGA CTAACTGTGTGGTATGAAGCCATTG 3′
XbaI Ter
Forward Primer for CAN 97-83 G Gene

[SEQ ID NO.:10]

5′ GGCGGCCGCCGTCGACGTTATGGAGGTGAAAGTAGAGAACA 3′
Sal I Met
Reverse Primer for CAN 97-83 G Gene

[SEQ ID NO.:11]

5′ GGCGGGTCTAGA CTAGTTTTGCATTGTGCTTACAGATG 3′
XbaI Ter
Forward Primer for CAN 98-75 G Gene

[SEQ ID NO.:12]

5′ GGCGGCCGCCGTCGACGCCATGGAAGCAAGAGTGGAGAACA 3′
Sal I Met
Reverse Primer for CAN 98-75 G Gene

[SEQ ID NO.:13]

5′ GGCGGGTCTAGA TTAACTACTTGGAGAAGATGTGTCTGTG 3′
XbaI Ter

To amplify the two G genes, Qiagen's OneStep RT-PCR Kit was used according to the manufacturer's instruction. Briefly, reverse transcription was carried out for 30 min at 50° C., followed by a 15 min incubation at 95° C. for the initial PCR activation step. Subsequently, 30 cycles of touch-down PCR were conducted to increase specificity of the reaction where denaturation was carried out at 94° C. for 1 min, initial annealing at 80° C. (and decreased by 0.5° C./cycle subsequently) for 1 min, and extension at 72° C. for 1.5 min. An additional 10 cycles of normal PCR were then carried out using an annealing temperature of 65° C., followed by a final extension at 72° C. for 10 min. Judged by the profile seen after agarose gel electrophoresis, a single specific DNA species of the right molecular size for the hMPV G gene (i.e. ˜690 bp from CAN 97-83 and ˜740 bp from CAN 98-75) was generated from each RNA template sample using the above PCR program. After being desalted with Qiagen's Qiaquick PCR Purification Kit, the PCR products were completely digested with Sal I and Xba I (New England Biolabs, Pickering, Ontario, Canada), and purified from agarose gel using Invitrogen's SNAP Gel Purification Kit (Burlington, Ontario, Canada).

For the amplification of the hMPV F gene, Qiagen's OneStep RT-PCR Kit proved unsatisfactory as Taq polymerase in the kit introduced multiple point mutations in the cDNA product generated. To overcome this, Qiagen's Sensiscript Reverse Transcriptase was combined with this company's ProofStart, version of Taq polymerase. The latter has proof-reading capabilities. The reverse transcription step was performed at 37° C. for 60 min, followed by an initial PCR activation step: a 5 min incubation at 95° C., 15 cycles of touch-down PCR: denaturation for 1 min at 94° C., initial annealing at 67.5° C. (with subsequent 0.5° C. reduction/cycle) for 1 min, and extension for 2 min at 72° C., 25 cycles of normal PCR: denaturation for 1 min at 94° C., annealing for 1 min at 60° C., and extension for 2 min at 72° C., and a final extension of 10 min at 72° C. This combination was satisfactory as it lead to the generation of a single cDNA of 1650 bp encoding the hMPV F protein. As for the PCR products for the hMPV G proteins, cDNA fragment for the F protein was desalted, digested with Sal I and Xba I, and gel-purified.

Molecular cloning of the full length F and G genes in VR-1012: Purified cDNA fragments encoding the conserved F and subtype-specific G proteins of hMPV were subcloned in VR-1012, a widely used DNA immunization vector developed by Vical Inc. (San Diego, Calif., US) (Coker et. al., 2003). It contains an expression cassette with transcription control elements, including the immediate early (IE) promoter and intron A sequences of the human cytomegalovirus (CMV), and the poly-A signal from human growth hormone (hGH) gene. The gene of interest with own initiation codon and Kozak sequence was cloned downstream of the CMV IE promoter and intron A, and upstream of the hGH poly-A site. The VR-1012 was digested with Sal I and Xba I, and then gel-purified, prior to being ligated to the above cDNA fragments. Electro-competent E. coli Top 10 cells (Invitrogen) were transformed. Plasmid mini-prep was used for initial screening where 3-5 clones/construct with the right molecular insert size between the Sal I and Xba I sites were then subjected to DNA sequencing of the entire hMPV genes.

Generation of truncated hMPV F and G gene Variants corresponding to secreted proteins using PCR: To compare the effectiveness of DNA vaccine vectors encoding the full membrane-anchored form of the hMPV F and G proteins with their deletion counterparts encoding secreted versions of the same protein, PCR using full-length, sequence-confirmed hMPV cDNA clones as templates and Qiagen's ProofStart was used to generate the latter. In essence, signal peptide at the N-terminus and the trans-membrane (TM) domain at the C-terminus were removed from the F protein via the PCR reaction. In contrast, intracellular and TM domains of the G proteins located at the N-termini of these typical type II glycoproteins were removed. Unique restriction enzyme sites at the end of the PCR fragments were also introduced for their convenient subsequent sub-cloning using the following PCR primers.

Forward Primer for CAN 97-83 F Gene (-Signal
Peptide; -TM Domain)

[SEQ ID NO.:14]

5′ GCCGCGGGATCCCTTAAAGAGAGCTACCTAGAAGAATC 3′
Bam HI
Reverse Primer for CAN 97-83 F Gene (-Signal
Peptide; -TM Domain)

[SEQ ID NO.:15]

5′ GCCGCGGGATCC CTAGCCAGTATTCCCTTTCTCTGCAC 3′
Bam HI Ter
Forward Primer for CAN 97-83 G Gene (-Intracell-
ular Domain; -TM Domain)

[SEQ ID NO.:16]

5′ GCCGCGGGATCCAACTACACAATACAAAAAACCTCATC 3′
Bam HI
Reverse Primer for CAN 97-83 G Gene (-Intracell-
ular Domain; -TM Domain)

[SEQ ID NO.:17]

5′ GCCGCGGGATCC CTAGTTTTGCATTGTGCTTACAGA 3′
Bam HI Ter
Forward Primer for CAN 98-75 G Gene (-Intracell-
ular Domain; -TM Domain)

[SEQ ID NO.:18]

5′ GCCGCGGGATCCGATCATGCAACATCAAAAAACATGACC 3′
Bam HI
Reverse Primer for CAN 98-75 G Gene (-Intracell-
ular Domain; -TM Domain)

[SEQ ID NO.:19]

5′ GCCGCGGGATCC TTAACTACTTGGAGAAGATGTGTCTG 3′
Bam HI Ter

Following the PCR reactions, molecular size, purity and yield of the products were determined using agarose gel electrophoresis. These DNA fragments were desalted, completely digested with Bam HI (New England Biolabs), and purified in gels as previously described.

Molecular cloning of the truncated genes encoding secreted F and G proteins of hMPV in VR-1020: VR1020, also developed by Vical Inc., was used to direct the expression of secreted proteins (Coker et. al., 2003). VR1020 has transcription control elements identical to those found in VR1012. In addition, it contains coding sequences for the signal peptide of human tissue plasminogen activator (TPA) downstream of the CMV IE promoter and intron A sequences and upstream of the hGH poly-A site. The gene of interest devoid of the authentic signal peptides was cloned downstream of the coding sequences for the signal peptide of TPA and upstream of the hGH poly-A site in VR1020. This insertion was made to be in frame with the TPA signal peptide, so that the latter could direct secretion of the expressed foreign protein. In this study, the PCR primers described in the previous section ensured in-frame insertion of the truncated hMPV genes in VR-1020. The vector was digested with Bam H1, treated with Antarctic Phosphatase (New England Biolabs) according to the manufacturer's instruction. The latter reagent was removed quickly by a spin column.

Purified cDNA fragments encoding truncated and secreted forms of the hMPV F and G proteins were ligated with the above VR-1020 vector, respectively. Transformation, screening and DNA sequencing of putative clones were performed as describe for the vectors made in VR-1012.

Vector construction to compare the authentic signal peptide in the F protein of hMPV with signal peptide from tissue plasminogen activator for DNA immunization: The following PCR primers were used to amplify the F gene of hMPV encoding a TM-truncated protein with intact authentic signal peptide using a full-length, sequence-confirmed hMPV F cDNA clone as the template, and Qiagen's ProofStart. The resulting PCR product was desalted, digested with Sal I and Bam HI, and purified using gels.

Forward Primer for CAN 97-83 F Gene (Authentic
Signal Peptide; -TM Domain)

[SEQ ID NO.:20]

5′ GGCGGCCGCCGTCGACAAAATGTCTTGGAAAGTGGTGATCA 3′
SalI Met
Reverse Primer for CAN 97-83 F Gene (Authentic
Signal Peptide; -TM Domain)

[SEQ ID NO.:21]

5′ GCCGCGGGATCC CTAGCCAGTATTCCCTTTCTCTGCAC 3′
Bam HI Ter

VR-1012 was digested with Sal I and Bam HI, gel-purified and ligated to the above PCR product. Transformation, screening and DNA sequencing were performed as described with the other vectors.

A list of the VR1012- and VR1020-based vectors generated for these studies is shown in Table 1.

TABLE 1
Vectors Made And Used And Designation Of Test Groups

| | | HMPV STRAIN | |
|---|---|---|---|
| | CLONE | HOMOLOGY | |
| TEST | DESIG- | (HMPV | |
| GROUP | NATION | SUBTYPE) | DESCRIPTION |
| 1 | VR1012 | None | Empty vector |
| 2 | Live hMPV | 26583 (A) | Live virus |
| 3 | VR1012 | 26583 (A) | Full-length, membrane |
| | 5-2 | | anchored F protein |
| 4 | VR1012 | 26583 (A) | Truncated, secreted |
| | 11-1 | | version of the F protein |
| | | | directed by the authentic |
| | | | signal peptide |
| 5 | VR1020 | 26583 (A) | Truncated, secreted |
| | 7-1 | | version of the F protein |
| | | | directed by the |
| | | | signal peptide of TPA |
| 6 | VR1012 | 26583 (A) | Full-length, membrane |
| | 2-4 | | anchored G protein |
| 7 | VR1012 | 26575 (B) | Full-length, membrane |
| | 3-4 | | anchored G protein |
| 8 | VR1020 | 26583 (A) | Truncated, secreted |
| | 8-2 | | version of the G protein |
| | | | directed by the |
| | | | TPA signal peptide |
| 9 | VR1020 | 26575 (B) | Truncated, secreted |
| | 9-1 | | version of the G protein |
| | | | directed by the |
| | | | TPA signal peptide |

Scale-up of plasmid DNA for studies in cotton rats: Upon DNA sequence confirmation of each hMPV gene in their appropriate vector, a correct clone was chosen from each construct, cultured in LB medium and purified using Qiagen's EndoFree Plasmid Giga Kits. Following the manufacturer's instructions, this kit efficiently reduced endotoxin to less than 0.1 EU/ug DNA. The purified DNA was quantified by both intensity comparison with standards on ethidium bromide-stained agarose gel as well as using absorbance reading at 260 nm (1 A₂₆₀ unit=50 μg/mL). There was an excellent agreement between the two measurements. Each final product was resuspended at the desired concentrations in endotoxin-free saline for injection into cotton rats.

Experimental design: The experiment evaluating the test vectors in cotton rats was performed twice. As a negative control in each experiment, the cotton rats in the first group in each experiment were lightly anesthetized with isoflurane and then inoculated intramuscularly (i.m.) with empty VR1012 vector (Group 1 in Table 1 and all figures). As a positive control in each experiment, the cotton rats in the second group in each experiment were lightly anesthetized with isoflurane and then inoculated intranasally (i.n.) with 1000 median cotton rat infectious doses (CRID₅₀; i.e., 10,000 median tissue culture infectious doses; TCID₅₀) of live hMPV 26583. These animals received no other inoculation during the course of the experiments. The remaining 7 groups of animals were similarly anesthetized and inoculated i.m. via the tabialis anterior (TA) muscle of both legs with one of the seven plasmid DNA vectors prepared as described above. The vectors were always suspended in endotoxin-free and nuclease-free saline. Each was administered in 0.2 ml volumes to the appropriate group three times, three weeks apart. In every instance, the dose of DNA in each inoculum was adjusted to have 100 μg DNA. Blood was obtained from each cotton rat just prior to the start of each experiment, immediately prior to each boosting inoculation and finally 21 days after the last inoculation. Sera was obtained from each blood sample, heat-inactivated at 56° C. for 30 min and then tested for hMPV-specific neutralizing antibodies against hMPV 26583 (Group A) as described above. The sera obtained from the last blood samples collected were also tested for their ability to neutralize the 26575 strain (Group B) of hMPV. After the last bleed, each cotton rat was anesthetized with isoflurane and then challenged i.n. with approximately 1000 CRID₅₀ of infectious hMPV 26583. Four days later, at a time that previous studies had indicated that peak virus titers in untreated animals administered this dose of virus occurred (Wyde et. al., 2005), each cotton rat was sacrificed and a nose wash and lung lavage fluid sample was obtained from them. These samples were assessed for hMPV lung virus titers as described above using either whole lungs or selected lobes as described above. To permit comparisons between animals of different weights and between lungs processed for virus, all lung titers were calculated on a per gram of lung tested basis.

Collection of nasal washes and lungs: Cotton rats were sacrificed using CO₂. The lungs of these animals were then removed, rinsed in sterile PBS (pH 7.2), weighed and transpleurally lavaged as described previously (Wilson et. al., 1980). Next, each cotton rat was decapitated and the lower jaw from each head disarticulated. Nose washes (NW) were collected by pushing 1 ml of MEM+2% FCS through each naris and capturing the effluent from the posterior opening of the palate.

Virus quantification: Levels of virus in different preparations were determined by serially diluting each sample in duplicate or quadruplicate in sterile 96-well tissue culture plates (Falcon 3072) using half log₁₀ dilutions as described previously (Wyde et. al., 2003 and Wyde et. al., 2005). These plates were incubated in a 5% CO₂ incubator maintained at 37° C. for 14 days. The medium in each well of the plates was replaced with fresh MEM-FCS+WT on day 5 of the assay. The monolayers in the wells of these plates were observed daily and scored for virus-induced cytopathic effects (CPE). Last readings for CPE formation were made on Day 14. At that time, the wells that were positive or negative for virus-induced CPE in each replicate row were noted. These data, the dilution of virus in the last wells exhibiting CPE and the interpolation method of Karber (Rhodes and Van Rooyen, 1953) were utilized to estimate the amount of virus present in the original suspension. Titers of virus pools, NW and lung lavage fluids (LF) were expressed as median tissue culture infectious doses (TCID₅O/ml; log₁₀). For virus pools, the minimum detectable virus concentration was 1.8 log₁₀TCID₅₀/ml. For NW and LF, the minimal detectable titers were 1.4 and 2.1 log₁₀ TCID₅O/ml, respectively.

Assessment of hMPV-specific neutralizing antibodies in sera: To obtain sera for antibody studies, animals were anesthetized with Isoflurane and then bled from the retro-orbital sinus plexus. Sera was prepared from each sample, heat inactivated at 56° C. for 30 minutes and then stored at 4° C. until assayed for virus-specific neutralizing antibodies in sterile 96-well tissue culture plates (Falcon 3072). The assay was performed as described in detail elsewhere (Wyde et. al., 1995), with three modifications. One, confluent monolayers of LLC-MK2 cells were utilized in these assays. Secondly, after serially diluting the sera, approximately 100 TCID₅₀ of the appropriate hMPV strain was added to the test and virus control wells. Finally, the morning after setting up an assay, the medium in each well of each test plate was removed and the cell monolayers in them were rinsed with PBS. Two hundred μL of MEM-FCS+WT was then added back to each well and the plates were returned to the 37° C. incubator. The cell monolayers in the virus control wells were observed daily. When these monolayers exhibited distinct virus-induced CPE, all of the wells in the assay were observed and scored for the presence or absence of virus. Titers were expressed as log₂ of the reciprocal of the last dilution of antiserum that completely inhibited virus-induced CPE. The minimum detectable virus neutralization antibody titer possible in these assays was 2.0 log₂/0.05 ml sera. It should be noted that undiluted sera from uninfected animals frequently “non-specifically” inhibited hMPV.

Statistics: Instat, a statistical program designed for IBM compatible computers (version 3, Graphpad Software, Inc., San Diego, Calif.) was used to calculate all means and standard deviations, as well as to perform the non-parametric analysis of variance (ANOVA) tests used to compare the different mean virus and virus-specific neutralizing antibody titers obtained in each experiment. For the purpose of statistical analyses, all values falling below the detection limits of an assay were assigned a value equivalent to that one well below the detection limit of the assay (e.g., in the TCID₅₀ assay for the determination of titers of virus in lungs, 1.7 log₁₀/ml was utilized since the limit of this assay was 2.1 log₁₀/ml).

### DETAILED FIGURE LEGENDS

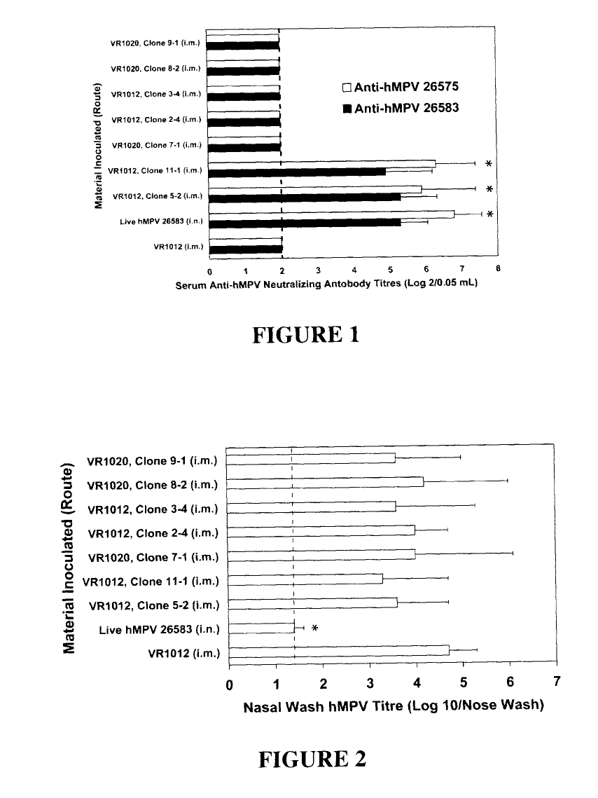

FIG. 1: Mean hMPV 26583 and 26575-specific neutralizing antibody titers (log₂) seen on day 63 (relative to the first inoculation and just prior to virus challenge) in the sera of cotton rats inoculated once with live hMPV 26583 intranasally (i.n.), three times, three weeks apart intramuscularly (i.m.) with empty plasmid or three times, three weeks apart, i.m. with one of the plasmid constructs listed to the left of the graph. The end of each bar represents the mean titer and the capped bars the standard deviation of each mean. The minimal detection limit in this assay was 2.0 log₂ (delineated by the vertical dashed line in the figure). The asterisk indicates statistical significance (p<0.05) when the demarcated mean was compared to the mean titer obtained for the negative control group (i.e., the group administered the empty VR1012 vector) using a non-parametric ANOVA. The number of cotton rats per group=7. Please see Table 1 for detailed description of each DNA vector.

FIG. 2: Mean titer of human metapneumovirus (hMPV) 26583 detected in nose washes of the cotton rats contained in each test group on day 4 post virus inoculation (67 days after these animals were inoculated once with live hMPV 26583 intranasally (i.n.), three times intramuscularly (i.m.) with empty plasmid or three times i.m. with one of the plasmid constructs listed to the left of the graph). The end of each bar represents the mean virus titer and the capped bars the standard deviation of each mean. The minimal detection limit in this assay was 1.4 log₁₀/nose wash (delineated by the vertical dashed line in the figure). The asterisk indicates statistical significance (p<0.05) when the demarcated mean was compared to the mean titer obtained for the negative control group (i.e., the group administered the empty VR1012 vector) using a non-parametric ANOVA. The number of cotton rats per group=7. Please see Table 1 for detailed description of each DNA vector.

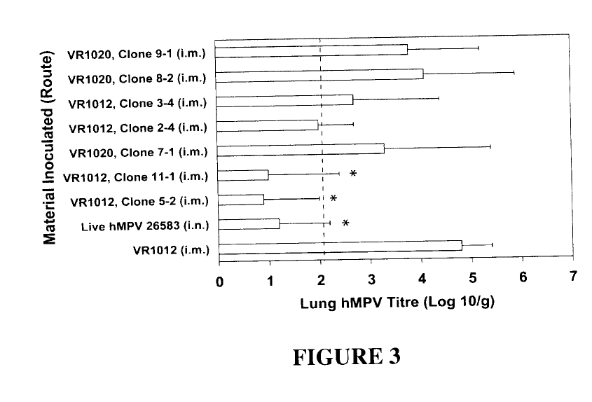

FIG. 3: Mean titer of human metapneumovirus (hMPV) 26583 detected in lungs of the cotton rats contained in each test group on day 4 post virus inoculation (67 days after these animals were inoculated once with live hMPV 26583 intranasally (i.n.), three times intramuscularly (i.m.) with empty plasmid or three times i.m. with one of the plasmid constructs listed to the left of the graph). The end of each bar represents the mean virus titer and the capped bars the standard deviation of each mean. The minimal detection limit in this assay was 2.1 log₁₀/g lung (delineated by the vertical dashed line in the figure). The asterisk indicates statistical significance (p<0.05) when the demarcated mean was compared to the mean titer obtained for the negative control group (i.e., the group administered the empty VR1012 vector) using a non-parametric ANOVA The number of cotton rats per group=7. Please see Table 1 for detailed description of each DNA vector.

Results and Discussion

Virus-specific neutralizing antibody serum titers: FIG. 1 displays the mean hMPV 26583- and 26575-specific neutralizing antibody titers detected on day 63 (relative to the first inoculation and just prior to virus challenge) in the sera of cotton rats inoculated three times, three weeks apart, with empty plasmid i.m.; inoculated once with live hMPV 26583 i.n.; or three times, three weeks apart, i.m. with one of the experimental plasmid constructs.

As the lengths of the bars in FIG. 1 indicate, the maximal mean hMPV-specific serum neutralizing antibody seen in this experiment occurred in the groups of cotton rats inoculated once with live hMPV, or three times with either clones 5-2 or 11-1 containing DNA for the production of full length, membrane-anchored, and a secreted hMPV F protein, respectively. The mean titers for these groups against hMPV 26583 were 5.3±0.8 log₂/0.05 ml, 5.3±1.0 log₂/0.05 ml and 4.9±1.3 log₂/0.05 ml, respectively. Their titres against hMPV 26575 were 6.8±0.8 log₂/0.05 ml, 5.9±1.5 log₂/0.05 ml and 6.3±1.1 log₂/0.05 ml, respectively. These titers were statistically indistinguishable from one another, and different (p<0.05) from the mean hMPV-specific neutralizing antibody titer determined for the group of cotton rats administered the empty vector VR1012 three times i.m. (i.e., 2.0±0.0 log₂) when compared using a non-parametric ANOVA. None of the other test groups had mean serum virus-specific neutralizing antibody titers that were significantly different from the mean serum titer detected in the negative control group.

Levels of hMPV in nose washes 4 days post virus challenge: FIG. 2 displays the mean titer of hMPV determined for the NW collected from the animals in each test group four days post virus challenge i.n. with 1000 CRID₅₀ of hMPV 26583. As the length of the bars in this figure indicate, with only one exception, the mean virus titers for the NW obtained for these animals all ranged between 3.3±1.2 log₁₀/nose wash (this being the mean virus titer in the group administered the DNA vector clone 11-1 encoding a truncated, secreted version of the hMPV F protein) and 4.7±0.7 log₁₀/nose wash (this being the mean virus titer for the negative control group). The single exception was the mean hMPV titer obtained for the group of cotton rats inoculated i.n. with live virus. This mean was 1.4±0.2 log₁₀/NW, the absolute minimal detection limit of the assay utilized to detect virus in the NW. When the different mean NW virus titers were compared utilizing the non-parametric ANOVA, only this last mean virus titer had a p value <0.05; its p value was <0.001.

Levels of hMPV in lungs 4 days post virus challenge: FIG. 3 displays the mean hMPV titers ascertained for the lungs of each test group of cotton rats four days after these animals were challenged i.n. with 1000 CRID₅₀ of hMPV 26583. As the length of the bars in this figure indicate, the mean virus titer measured in the lungs of animals ranged between 0.9±1.1 log₁₀/g lung (the mean virus titre obtained for the group of animals administered clone 5-2, the vector containing the DNA for the production of full length, membrane-anchored, hMPV F protein) and 4.8±0.6 log₁₀/g lung (the mean virus titre in the lungs of the negative control group). Five of the 8 test groups had >2 log₁₀/g lung reductions in mean pulmonary virus titer compared to the mean virus lung titer measured in the negative control group: 1) the group administered live virus once i.n. (mean lung virus titer=1.2±1.0 log₁₀/g lung); 2) the group administered vector clone 5-2 (the DNA vector encoding full-length, membrane-anchored hMPV F protein), mean virus lung titer=0.9±1.1 log₁₀/g lung); 3) the group administered vector clone 11-1 (the DNA vector encoding a truncated, secreted F protein of hMPV 26583 directed by the authentic signal peptide, mean virus lung titer=1.0±1.4 log₁₀/g lung); 4) the group inoculated thrice with vector clone 2-4 (the DNA vector encoding full-length, membrane anchored G protein of hMPV 26583, 2.0±0.7 log₁₀/g lung) and 5) the cotton rats administered clone 3-4 (the vector encoding full-length, membrane anchored G protein of hMPV 26575, 2.7±1.7 log₁₀/g lung). However, only the mean virus titers in the lungs of the first three of these groups were statistically significantly reduced when the means of these groups of animals were compared to the mean hMPV lung virus titer determined for the negative control group (the p value obtained for all three groups using the non-parametric ANOVA being <0.05).

As the results in FIG. 1 show, virus-specific neutralizing antibody responses were induced in the test animals, which received 3 doses of 100 μg plasmid DNA/dose. Specifically, the groups of cotton rats inoculated with clone 5-2 (i.e., the DNA vector encoding full-length, membrane anchored F protein of hMPV subgroup A) and 11-1 (i.e. the DNA vector encoding a truncated, secreted version of the F protein, directed by the authentic signal peptide) mounted statistically significant neutralizing antibody responses against both hMPV subgroups. The mean neutralizing antibody titers of these animals for the subgroup A hMPV (5.3±1.0 log₂/0.05 mL, and 4.9±1.3 log₂/0.05 mL, respectively) were statistically equivalent to those seen in the group of cotton rats that were inoculated on Day 0 with live hMPV (5.3±0.8 log₂/0.05 mL). A similar observation is made for neutralizing antibody titres for the subgroup B hMPV (5.9±1.5 log₂/0.05 mL and 6.3±1.1 log₂/0.05 mL for animals received vectors 5-2 and 11-1, respectively, versus 6.8±0.8 log₂/0.05 mL for animals inoculated with live hMPV). Moreover, the animals in these groups demonstrated equivalent protection of their lower respiratory tracts as the cotton rats inoculated with the live virus (FIG. 3; 0.9-1.0±1.1-1.4 log₁₀/g lung vs 1.2±1.0 log₁₀/g lung). This indicates that serum virus neutralizing antibody titre is likely the primary immune correlate of protection in this animal model and inversely correlates with lung virus titre post challenge (r=0.7).

Interestingly, only the animals inoculated with live virus were protected from hMPV infection of the upper respiratory tract (as indicated by nose wash titers; FIG. 2; 1.4±0.2 log₁₀/nose wash vs 4.8±0.2 log₁₀/nose wash for the negative control). This is likely due to the low mucosal immune response induced by the DNA vaccine vectors given by the parental i.m. route, in contrast to the high local immune response in animals that received i.n. virus inoculation, a phenomenon which has been observed with a number of other respiratory viruses.

Although clones 5-2 and 11-1 were derived from hMPV subgroup A virus, we expect animals received them to be protected against subgroup B hMPV infection of the lung for the following reasons: 1). the F protein is conserved between the two virus subgroups; 2). strong neutralizing activity against the subgroup B virus (i.e. 26575) was observed in animals received these clones, respectively, which were statistically indistinguishable from animals received live hMPV.

As can be understood by one skilled in the art, many modifications to the exemplary embodiments described herein are possible. The invention, rather, is intended to encompass all such modification within its scope, as defined by the claims.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### REFERENCES

1. Ada G, Ramshaw I. (2003) DNA vaccination. Expert Opin. Emerg. Drugs 8(1):27-35.
2. Bastien N, Normand S, Taylor T, Ward D, Peret T C, Boivin G, Anderson L J, Li Y. (2003) Sequence analysis of the N, P, M and F genes of Canadian human metapneumovirus strains. Virus Res. 93(1):51-62.
3. Biacchesi S, Pham Q N, Skiadopoulos M H, Murphy B R, Collins P L, Buchholz U J. (2005) Infection of nonhuman primates with recombinant human metapneumovirus lacking the SH, G, or M2-2 protein categorizes each as a nonessential accessory protein and identifies vaccine candidates. J. Virol. 79(19):12608-13.
4. Biacchesi S, Skiadopoulos M H, Boivin G, Hanson C T, Murphy B R, Collins P L, Buchholz U J. (2003) Genetic diversity between human metapneumovirus subgroups. Virology 315(1):1-9.
5. Biacchesi S, Skiadopoulos M H, Yang L, Lamirande E W, Tran K C, Murphy B R, Collins P L, Buchholz U J. (2004) Recombinant human Metapneumovirus lacking the small hydrophobic SH and/or attachment G glycoprotein: deletion of G yields a promising vaccine candidate. J. Virol. 78(23):12877-87.
6. Chambers R S, Johnston S A. (2003) High-level generation of polyclonal antibodies by genetic immunization. Nat. Biotechnol. 21(9):1088-92.
7. Coker C, Majid M, Radulovic S. (2003) Development of Rickettsia prowazekii DNA vaccine: cloning strategies. Ann. N.Y. Acad. Sci. 990:757-64.
8. Crowe, J. E., Jr. (1995) Current approaches to the development of vaccines against disease caused by respiratory syncytial virus (RSV) and parainfluenza virus (PIV). Vaccine. 13:415-421.
9. Ebihara T, Endo R, Kikuta H, Ishiguro N, Yoshioka M, Ma X, Kobayashi K. (2003) Seroprevalence of human metapneumovirus in Japan. J. Med. Virol. 70(2):281-3.
10. Englund J A, Champlin R E, Wyde P R, Kantarjian H, Atmar R L, Tarrand J, Yousuf H, Regnery H, Klimov A I, Cox N J, Whimbey E. (1998) Common emergence of amantadine- and rimantadine-resistant influenza A viruses in symptomatic immunocompromised adults. Clin. Infect. Dis. 26(6): 1418-24.
11. Falsey A R, Erdman D, Anderson L J, Walsh E E. (2003) Human metapneumovirus infections in young and elderly adults. J. Infect. Dis. 187(5):785-90.
12. Freymouth F, Vabret A, Legrand L, Eterradossi N, Lafay-Delaire F, Brouard J, Guillois B. (2003) Presence of the new human metapneumovirus in French children with bronchiolitis. Pediatr. Infect. Dis. J. 22(1):92-4.
13. Greensill J, McNamara P S, Dove W, Flanagan B, Smyth R L, Hart C A. (2003) Human metapneumovirus in severe respiratory syncytial virus bronchiolitis. Emerg. Infect. Dis. 9(3):372-5.
14. Guiducci C, Ott G, Chan J H, Damon E, Calacsan C, Matray T, Lee K-D, Coffman R L, Barrat F J. (2006) Properties regulating the nature of the plasmacytoid dendritic cell response to Toll-like receptor 9 activation. J. Exp. Med. 203 (8):1999-2008.
15. Hamelin M E, Yim K, Kuhn K H, Cragin R P, Boukhvalova M, Blanco J C, Prince G A, Biovin G. (2005) Pathogenesis of human metapneumovirus lung infection in BALB/c mice and cotton rats. J. Virol. 79(14): 8894-903.
16. Herd K A, Mahalingam S, Mackay I M, Nissen M, Sloots T P, Tindle R W (2006) Cytotoxic T-lymphocyte epitope vaccination protects against human metapneumovirus infection and disease in mice. J. Virol. 80(4):2034-44.
17. Jartti T, van den Hoogen B, Garofalo R P, Osterhaus A D, Ruuskanen O. (2002) Metapneumovirus and acute wheezing in children. Lancet 360(9343):1393-4.
18. Johnson P R Jr, Olmsted R A, Prince G A, Murphy B R, Alling D W, Walsh E E, Collins P L. (1987a) Antigenic relatedness between glycoproteins of human respiratory syncytial virus subgroups A and B: evaluation of the contributions of F and G glycoproteins to immunity. J. Virol. 61(10):3163-6.
19. Johnson P R, Spriggs M K, Olmsted R A, Collins P L. (1987b) The Glycoprotein of human respiratory syncytial viruses of subgroups A and B: extensive sequence divergence between antigenically related proteins. Proc. Natl. Acad. Sci. USA. 84(16):5625-9.
20. Johnson P R, Collins P L. (1988) The fusion glycoproteins of human respiratory syncytial virus of subgroups A and B: sequence conservation provides a structural basis for antigenic relatedness. J. Gen. Virol. 69(10):2623-8.
21. Kapczynski D R, Sellers H S. (2003) Immunization of turkeys with a DNA vaccine expressing either the F or N gene of avian metapneumovirus. Avian Dis. 47(4): 1376-83.
22. Leclercq S, Harms J S, Oliveira S C. (2003) Enhanced efficacy of DNA vaccines against an intracellular bacterial pathogen by genetic adjuvants. Curr. Pharm. Biotechnol. 4(2):99-107
23. Li X, Sambhara S, Li C X, Ewasyshyn M, Parrington M, Caterini J, James O, Cates G, Du R P, Klein M. (1998) Protection against respiratory syncytial virus infection by DNA immunization. J. Exp. Med. 188(4):681-8.
24. Li X, Sambhara S, Li C X, Ettorre L, Switzer I, Cates G, James O, Parrington M, Oomen R, Du R P, Klein M. (2000) Plasmid DNA encoding the respiratory syncytial virus G protein is a promising vaccine candidate. Virology 269(1):54-65.
25. Maggi F, Pifferi M, Vatteroni M, Formai C, Tempestini E, Anzilotti S, Lanini L, Andreoli E, Ragazzo V, Pistello M, Specter S, Bendinelli M. (2003) Human metapneumovirus associated with respiratory tract infections in a 3-year study of nasal swabs from infants in Italy. J. Clin. Microbiol. 41(7):2987-91.
26. Mejias A, Chavez-Bueno S, Ramilo O. (2004) Human metapneumovirus: a not so new virus. Pediatr. Infect. Dis. J. 23(1):1-7.
27. Nissen M D, Siebert D J, Mackay I M, Sloots T P, Withers S J. (2002) Evidence of human metapneumovirus in Australian children. Med. J. Aust. 176(4):188.
28. Peret T C, Boivin G, Li Y, Couillard M, Humphrey C, Osterhaus A D, Erdman D D, Anderson L J. (2002) Characterization of human metapneumoviruses isolated from patients in North America. J. Infect. Dis. 185(11):1660-3.
29. Pham Q N, Biacchesi S, Skiadopoulos M H, Murphy B R, Collins P L, Buchholz U J. (2005) Chimeric recombinant human metapneumoviruses with the nucleoprotein or phosphoprotein open reading frame replaced by that of avian metapneumovirus exhibit improved growth in vitro and attenuation in vivo. J. Virol. 79(24): 15114-22.
30. Plotnicky-Gilquin H, Robert A, Chevalet L, Haeuw J F, Beck A, Bonnefoy J Y, Brandt C, Siegrist C A, Nguyen T N, Power U F. (2000) CD4(+) T-cell-mediated antiviral protection of the upper respiratory tract in BALB/c mice following parenteral immunization with a recombinant respiratory syncytial virus G protein fragment. J Virol. 74:3455-3463.
31. Polack F P, Karron R A. (2004) The future of respiratory syncytial virus vaccine development. Pediatr. Infect. Dis. J. (1 Suppl):S65-73.
32. Raviprakash K, Ewing D, Simmons M, Porter K R, Jones T R, Hayes C G, Stout R, Murphy G S. (2003) Needle-free Biojector injection of a dengue virus type 1 DNA vaccine with human immunostimulatory sequences and the GM-CSF gene increases immunogenicity and protection from virus challenge in Aotus monkeys. Virology 315(2):345-52.
33. Rhodes, A. J. and Van Rooyen, C. E. (1953). Textbook of Virology, pp. 66-69. Williams and Wilkins, Baltimore, Md.
34. Siddiqui A A, Phillips T, Charest H, Podesta R B, Quinlin M L, Pinkston J R, Lloyd J D, Paz M, Villalovos R M, Pompa J. (2003) Induction of protective immunity against Schistosoma mansoni via DNA priming and boosting with the large subunit of calpain (Sm-p80): adjuvant effects of granulocyte-macrophage colony-stimulating factor and interleukin-4. Infect. Immun. 71(7):3844-51.
35. Srivastava I K, Liu M A. (2003) Gene vaccines. Ann. Intern. Med. 138(7):550-9.
36. St Clair N, Shenoy B, Jacob L D, Margolin A L. (1999) Cross-linked protein crystals for vaccine delivery. Proc. Natl. Acad. Sci. USA. 96(17):9469-74.
37. Tang R S, Mahmood K, Macphail M, Guzzetta J M, Haller A A, Liu H, Kaur J, Lawlor H A, Stillman E A, Schickli J H, Fouchier R A, Osterhaus A D, Spaete R R. (2005) A host-range restricted parainfluenza virus type 3 (PIV3) expressing the human metapneumovirus (hMPV) fusion protein elicits protective immunity in African green monkeys. Vaccine 23(14):1657-67.
38. Temperton N J, Quenelle D C, Lawson K M, Zuckerman J N, Kern E R, Griffiths P D, Emery V C. (2003) Enhancement of humoral immune responses to a human cytomegalovirus DNA vaccine: adjuvant effects of aluminum phosphate and CpG oligodeoxynucleotides. J. Med. Virol. 70(1): 86-90.
39. Valenzuela P, Medina A, Rutter W J, Ammerer G, Hall B D. (1982) Synthesis and assembly of hepatitis B virus surface antigen particles in yeast. Nature 298(5872):347-50.
40. van den Hoogen B G, de Jong J C, Groen J, Kuiken T, de Groot R, Fouchier R A, Osterhaus A D. (2001) A newly discovered human pneumovirus isolated from young children with respiratory tract disease. Nat. Med. 7(6):719-24.
41. Viazov S, Ratjen F, Scheidhauer R, Fiedler M, Roggendorf M. (2003) High prevalence of human metapneumovirus infection in young children and genetic heterogeneity of the viral isolates. J. Clin. Microbiol. 41(7):3043-5.
42. Vicente D, Cilla G, Montes M, Perez-Trallero E. (2003) Human metapneumovirus and community-acquired respiratory illness in children. Emerg. Infect. Dis. 9(5):602-3.
43. Vollmer J, Weeratna R, Payette P, Jurk M, Schetter C, Laucht M, Wader T, Tluk S, Liu M, Davis H L, Krieg A M. (2004) Characterization of three CpG oligodeoxynucleotide classes with distinct immunostimulatory activities. Eur. J. Immunol. 34(1):251-62.
44. Wertz G W, Collins P L, Huang Y, Gruber C, Levine S, Ball L A. (1985) Nucleotide sequence of the G protein gene of human respiratory syncytial virus reveals an unusual type of viral membrane protein. Proc. Natl. Acad. Sci. USA. 82(12):4075-9.
45. Wilkesmann A, Schildgen O, Eis-Hubinger A M, Geikowski T, Glatzel T, Lentze M J, Bode U, Simon A. (2006) Human metapneumovirus infections cause similar symptoms and clinical severity as respiratory syncytial virus infections. Eur. J. Pediatr. (details to be added when available)
46. Williams J V, Crowe J E Jr, Enriquez R, Minton P, Peebles R S Jr, Hamilton R G, Higgins S, Griffin M, Hartert T V. (2005) Human metapneumovirus infection plays an etiologic role in acute asthma exacerbations requiring hospitalization in adults. J. Infect. Dis. 192(7): 1149-53.
47. Williams J V, Harris P A, Tollefson S J, Halburnt-Rush L L, Pingsterhaus J M, Edwards K M, Wright P F, Crowe J E Jr. (2004) Human metapneumovirus and lower respiratory tract disease in otherwise healthy infants and children. N. Engl. J. Med. 350(5):443-50.
48. Wilson, S. Z., Knight, V., Wyde, P. R., Drake, S., and Couch, R. B. (1980). Amantadine and ribavirin aerosol treatment of influenza A and B infection in mice. Antimicrob. Agents and Chemother. 17, 642-648.
49. Wyde P R. (1998) Respiratory syncytial virus (RSV) disease and prospects for its control. Antiviral Res. 39(2):63-79.
50. Wyde P R. (1999) Chemotherapy of respiratory viruses: prospects and challenges. Drug Resist. Update 2(4):244-258.
51. Wyde P R, Moore-Poveda D K, De Clercq E, Neyts J, Matsuda A, Minakawa N, Guzman E, Gilbert B E. (2000a) Use of cotton rats to evaluate the efficacy of antivirals in treatment of measles virus infections. Antimicrob. Agents Chemother. 44(5): 1146-52.
52. Wyde P R, Stittelaar K J, Osterhaus A D, Guzman E, Gilbert B E. (2000b) Use of cotton rats for preclinical evaluation of measles vaccines. Vaccine 19(1):42-53.
53. Wyde P R, Chetty S N, Jewell A M, Boivin G, Piedra P A. (2003) Comparison of the inhibition of human metapneumovirus and respiratory syncytial virus by ribavirin and immune serum globulin in vitro. Antiviral Res. 60(1):51-9.
54. Wyde P R, Cetty S N, Jewell A M, Schoonover S L, Piedra P A. (2005) Development of a cotton rat-human metapneumovirus (hMPV) model for identifying and evaluating potential hMPV antivirals and vaccines. Antiviral Res. 66(1): 57-66.
55. Zhang Y, Wang Y, Gilmore X, Xu K, Wyde P R, Mbawuike I N. (2002) An aged mouse model for RSV infection and diminished CD8(+) CTL responses. Exp. Biol. Med. (Maywood). 227(2):133-40.

1. An immunological composition comprising a recombinant nucleic acid vector, the nucleic acid vector comprising a promoter region operably linked to a coding sequence encoding a human metapneumovirus F antigen or a human metapneumovirus G antigen and a pharmaceutically acceptable carrier.

2. The immunological composition of claim 1 wherein the promoter region comprises human CMV immediate early promoter, SV40 promoter, desmin promoter/enhancer, creatine kinase promoter, metallothionein promoter, 1,24-vitaminD(3)(OH)(2) dehydroxylase promoter or Rous Sarcoma Virus long terminal repeat.

3. (canceled)

4. The immunological composition of claim 1 wherein the coding sequence encodes the human metapneumovirus F antigen.

5. The immunological composition of claim 4 wherein the coding sequence encoding the human metapneumovirus F antigen: (i) comprises the sequence of any one of SEQ ID NOS: 1 to 3; (ii) consists of the sequence of any one of SEQ ID NOS: 1 to 3; (iii) consists of a sequence having at least 95% identity to the sequence of any one of SEQ ID NOS: 1 to 3; or (iv) consists of at least 8 amino acids of the sequence of any one of SEQ ID NOS: 1 to 3.

6. (canceled)

7. (canceled)

8. (canceled)

9. The immunological composition of claim 1 wherein the coding sequence encodes the human metapneumovirus G antigen.

10. The immunological composition of claim 9 wherein the coding sequence encoding the human metapneumovirus G antigen; (i) comprises the sequence of any one of SEQ ID NOS: 4 to 7; (ii) consists of the sequence of any one of SEQ ID NOS: 4 to 7; (iii) Consists of a sequence having at least 95% identity to the sequence of any one of SEQ ID NOS: 4 to 7; or (iv) consists of at least 8 amino acids of the sequence of any one of SEQ ID NOS: 4 to 7.

11. (canceled)

12. (canceled)

13. (canceled)

14. The immunological composition of claim 1 further comprising an enhancer element operably linked to the promoter region.

15. The immunological composition of claim 14 wherein the enhancer element comprises human CMV enhancer, SV40 enhancer, alpha-fetoprotein enhancer or tyrosinase enhancer.

16. (canceled)

17. The immunological composition of claim 1 further comprising an intronic sequence operably linked to the promoter region and the coding sequence.

18. The immunological composition of claim 17 wherein the intronic sequence is intron A from human CMV or rabbit β-globin intron II.

19. The immunological composition of claim 1 further comprising a polyadenylation signal downstream of, and operably linked to, the coding sequence.

20. The immunological composition of claim 19 wherein the polyadenylation signal comprises SV40 polyadenylation signal, rabbit β-globin polyadenylation signal, bovine growth hormone polyadenylation signal or human growth hormone polyadenylation signal.

21. (canceled)

22. The immunological composition of claim 1 further comprising an adjuvant.

23. The immunological composition of claim 22 wherein the adjuvant comprises Freund's complete adjuvant solution, Freund's incomplete adjuvant solution, a fatty acid, a monoglyceride, a protein, a carbohydrate, aluminium oxide, a toxin, a killed microbe, ethylene-vinyl acetate copolymer, L-tyrosine, manide-oleate, an immunostimulatory nucleic acid sequence or a nucleic acid encoding a protein.

24. (canceled)

25. (canceled)

26. (canceled)

27. (canceled)

28. The immunological composition of claim 1 wherein the nucleic acid vector is a DNA plasmid.

29. The immunological composition of claim 1 that is formulated for injection.

30. (canceled)

31. The immunological composition of claim 30 wherein the carrier comprises liposomes or particles for use with a gene gun.

32. A method of eliciting an immune response to human metapneumovirus in an individual, comprising administering an effective amount of the immunological composition defined in claim 1 to the individual.

33. The method of claim 32 wherein the individual is a human.

34. The method of claim 32 wherein the nucleic acid vector is a DNA plasmid and from about 0.1 g to about 1000 μg of the DNA plasmid is administered to the individual.

35. The method of claim 32 wherein a priming dose of the immunological composition is administered to the individual followed by administration of a boost dose to the individual.

36. The method of claim 32 wherein the immunological composition is administered by injection.

37. The method of claim 32 further comprising administering an adjuvant to the individual.

38. The method of claim 37 wherein the adjuvant comprises Freund's complete adjuvant solution, Freund's incomplete adjuvant solution, a fatty acid, a monoglyceride, a protein, a carbohydrate, aluminium oxide, a toxin, a killed microbe, ethylene-vinyl acetate copolymer L-tyrosine, manide-oleate, an immunostimulatory nucleic acid sequence or a nucleic acid encoding a protein.

39. (canceled)

40. A method for producing an antibody specific against a human metapneumovirus F antigen or a human metapneumovirus G antigen comprising administering an effective amount of the immunological composition defined in claim 31 to an individual; and isolating an antibody or an immune cell from the individual, the antibodies or immune cell specific against the human metapneumovirus F antigen or human metapneumovirus G antigen.


SEQUENCE LISTING (21 sequences)

SEQ ID NO: 1 - protein, 539 aa, hMPV
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 2 - protein, 490 aa, hMPV
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 3 - protein, 472 aa, hMPV
LKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 4 - protein, 219 aa, hMPV
MEVKVENIRAIDMLKARVKNRVARSKCFKNASLILIGITTLSIALNIYLIINYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 5 - protein, 236 aa, hMPV
MEARVENIRAIDMFKAKMKNRIRSSKCHRNATLILIGSTAPSMALNTLLIIDHATSKNMTKVEHCVNMPPVEPSKKTPMTSAADPNTKPNPQQATQLTTEDSTSLAATLEDHLHTGTTPTPDATVSQQTTDEHTTLLRSTNRQTTQTTAEKKPTRATTKKETTTRTTSTAATQTLNTTNQTSNGREATTTSARSRNNATTQSSDQTTQAADPSSQSQHTQKSTTTTHNTDTSSPSS

SEQ ID NO: 6 - protein, 168 aa, hMPV
NYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 7 - protein, 185 aa, hMPV
DHATSKNMTKVEHCVNMPPVEPSKKTPMTSAADPNTKPNPQQATQLTTEDSTSLAATLEDHLHTGTTPTPDATVSQQTTDEHTTLLRSTNRQTTQTTAEKKPTRATTKKETTTRTTSTAATQTLNTTNQTSNGREATTTSARSRNNATTQSSDQTTQAADPSSQSQHTQKSTTTTHNTDTSSPSS

SEQ ID NO: 8 - DNA, 41 bp, artificial
  synthetic primer
ggcggccgccgtcgacaaaatgtcttggaaagtggtgatca

SEQ ID NO: 9 - DNA, 37 bp, artificial
  synthetic primer
ggcgggtctagactaactgtgtggtatgaagccattg

SEQ ID NO: 10 - DNA, 41 bp, artificial
  synthetic primer
ggcggccgccgtcgacgttatggaggtgaaagtagagaaca

SEQ ID NO: 11 - DNA, 38 bp, artificial
  synthetic primer
ggcgggtctagactagttttgcattgtgcttacagatg

SEQ ID NO: 12 - DNA, 41 bp, artificial
  synthetic primer
ggcggccgccgtcgacgccatggaagcaagagtggagaaca

SEQ ID NO: 13 - DNA, 40 bp, artificial
  synthetic primer
ggcgggtctagattaactacttggagaagatgtgtctgtg

SEQ ID NO: 14 - DNA, 38 bp, artificial
  synthetic primer
gccgcgggatcccttaaagagagctacctagaagaatc

SEQ ID NO: 15 - DNA, 38 bp, artificial
  synthetic primer
gccgcgggatccctagccagtattccctttctctgcac

SEQ ID NO: 16 - DNA, 38 bp, artificial
  synthetic primer
gccgcgggatccaactacacaatacaaaaaacctcatc

SEQ ID NO: 17 - DNA, 36 bp, artificial
  synthetic primer
gccgcgggatccctagttttgcattgtgcttacaga

SEQ ID NO: 18 - DNA, 39 bp, artificial
  synthetic primer
gccgcgggatccgatcatgcaacatcaaaaaacatgacc

SEQ ID NO: 19 - DNA, 38 bp, artificial
  synthetic primer
gccgcgggatccttaactacttggagaagatgtgtctg

SEQ ID NO: 20 - DNA, 41 bp, artificial; identical to SEQ ID NO: 8
  synthetic primer

SEQ ID NO: 21 - DNA, 38 bp, artificial; identical to SEQ ID NO: 15
  synthetic primer